(19) United States
(12) Patent Application Publication
(10) Pub. No.: US 2020/0390850 A1
(43) Pub. Date: Dec. 17, 2020

(54) PEPTIDES AND USES THEREOF

(71) Applicant: LATERAL IP PTY LTD, Melbourne, Victoria (AU)

(72) Inventors: Andrew GEARING, Melbourne, Victoria (AU); David KENLEY, Melbourne, Victoria (AU)

(73) Assignee: LATERAL IP PTY LTD, Melbourne, Victoria (AU)

(21) Appl. No.: 16/962,082

(22) PCT Filed: Jan. 15, 2019
(86) PCT No.: PCT/AU2019/050020

§ 371 Date: Jul. 14, 2020

(30) Foreign Application Priority Data
Jan. 15, 2018 (AU) 2018900117

(51) Int. Cl. A61K 38/10; A61K 38/08; A61K 45/06; A61K 9/00; A61P 29/00

(52) U.S. Cl. CPC A61K 38/10; A61K 38/08; A61P 29/00; A61K 9/0053; A61K 45/06

(57) ABSTRACT

A method of treating neuropathic pain in a subject, the method comprising administering to a subject a therapeutically effective amount of a peptide of formula (I) or (II), or a pharmaceutically acceptable salt thereof: R1-CRSVEG-SCG-R2 (I) (SEQ ID NO:1) wherein R1 is selected from the group consisting of YL-RIVQ, LRIVQ, RIVQ, IVQ, VQ, and Q, or R is absent; and R2 is F (phenylalanine) or R2 is absent. R1-CRRFVESSC-R2 (II) (SEQ ID NO:6) wherein R1 is selected from the group consisting of YLRVMIK, LRVMK, RVMK, VMK, MK, and K, or R1 is absent; and R2 is selected from the group consisting of A (alanie) and AF (alanine-phenylalanie), or R2 is absent.

### FIELD OF THE INVENTION

The invention relates to peptides useful in treating neuropathic pain, and methods of their use, including alleviating neuropathic pain or delaying the onset of neuropathic pain. Methods of producing analgesia are also described.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Neuropathic pain is caused by a primary lesion, malfunction or dysfunction in the peripheral or central nervous system. Unlike nociceptive pain, which serves a protective biological function by warning of tissue damage, neuropathic pain has no protective effect and can develop days or months after an injury or after resolution of a disease state, and is frequently long-lasting and chronic.

Neuropathic pain may result from nerve damage caused by a trauma such as a sporting injury, an accident, a fall or a penetrating injury or the nerve damage may result from a disease process such as stroke, viral infections, exposure to toxins, degenerative diseases and diabetes. The prevalence of disease states which may result in the development of neuropathic pain conditions, such as diabetic neuropathy and post-herpetic neuralgia, is increasing and therefore an increasing number of people are suffering chronic neuropathic pain symptoms.

Although there are effective remedies for treating nociceptive pain, neuropathic pain is often resistant to available analgesic drugs. In addition, current therapies such as tricyclic antidepressants, anticonvulsants, opioid and non-opioid analgesics have significant side effects such as sedation and sleepiness and in the case of opioid analgesics, the risk of drug tolerance and drug dependency or addiction. Moreover, there are currently few useful options available for the treatment of neuropathic pain in the absence of an analgesic effect on nociceptive pain. Accordingly, there remains an urgent need for new and alternative options that are effective for the selective treatment of neuropathic pain, with limited or no side effects. The present invention solves, or partly alleviates, this problem by providing compounds that are effective at alleviating neuropathic pain with minimal or no analgesic effect on nociceptive pain.

### SUMMARY OF THE INVENTION

In an aspect disclosed herein, there is provided a method of treating neuropathic pain in a subject, the method comprising administering to a subject a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In an embodiment, said therapeutically effective amount alleviates neuropathic pain in the subject in the absence of a therapeutically effective analgesic effect on nociceptive pain.

In an embodiment, the subject is a human.

In an embodiment, the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

In an embodiment, the method further comprises administering to the subject a therapeutically effective amount of a second agent capable of alleviating pain in the subject, wherein the second agent is not the peptide of formula (I) or a pharmaceutically acceptable salt thereof.

In another aspect disclosed herein, there is provided a pharmaceutical composition comprising a peptide of formula (I), or a pharmaceutically acceptable salt thereof, for the treatment of neuropathic pain in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In another aspect disclosed herein, there is provided use of a peptide of formula (I), or a pharmaceutically acceptable salt thereof, in the manufacture of a medicament for the treatment of neuropathic pain in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In another aspect disclosed herein, there is provided use of a pharmaceutical composition comprising:

(i) a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent; and
(ii) a second agent capable of alleviating pain in the subject, wherein the second agent is not the peptide of formula (I) or a pharmaceutically acceptable salt thereof.

In another aspect disclosed herein, there is provided an analgesic composition comprising a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In another aspect disclosed herein, there is provided a composition comprising a therapeutically effective amount of a peptide, or a pharmaceutically acceptable salt thereof, wherein the peptide consists of amino acid sequence CRSVEGSCG (SEQ ID NO:4) or amino acid sequence CRSVEGSCGF (SEQ ID NO:5).

In another aspect disclosed herein, there is provided a method of treating a condition in a subject, the method comprising administering to a subject a therapeutically effective amount of a peptide, or a pharmaceutically acceptable salt thereof, wherein the peptide consists, or consists essentially of, amino acid sequence CRSVEGSCG (SEQ ID NO:4) or amino acid sequence CRSVEGSCGF (SEQ ID NO:5), and wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, and a bone disorder.

In another aspect disclosed herein, there is provided a method of treating neuropathic pain in a subject, the method comprising administering to a subject a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

In an embodiment, the therapeutically effective amount alleviates neuropathic pain in the subject in the absence of a therapeutically effective analgesic effect on nociceptive pain.

In an embodiment, the subject is selected from the group consisting of a feline, a canine and an equine.

In an embodiment, the method further comprises administering to the subject a therapeutically effective amount of a second agent capable of alleviating pain in the subject, wherein the second agent is not the peptide of formula (II) or a pharmaceutically acceptable salt thereof.

In another aspect disclosed herein, there is provided a pharmaceutical composition comprising a peptide of formula (II), or a pharmaceutically acceptable salt thereof, for use in the treatment of neuropathic pain in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In another aspect disclosed herein, there is provided a use of a peptide of formula (II), or a pharmaceutically acceptable salt thereof, in the manufacture of a medicament for the treatment of neuropathic pain in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In another aspect disclosed herein, there is provided a pharmaceutical composition comprising:

(i) a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent, and
(ii) a second agent capable of alleviating pain in the subject, wherein the second agent is not the peptide of formula (II) or a pharmaceutically acceptable salt thereof.

In another aspect disclosed herein, there is provided an analgesic composition comprising a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In another aspect disclosed herein, there is provided a use of a peptide of formula (II), or a pharmaceutically acceptable salt thereof, in the manufacture of a medicament for the treatment of neuropathic pain in a subject.

In another aspect disclosed herein, there is provided a method of treating a condition in a subject, the method comprising administering to a subject a therapeutically effective amount of a composition comprising a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent, and
wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, and a bone disorder.

### BRIEF DESCRIPTION OF THE FIGURES

FIG. 1 is a schematic diagram showing the preparation of spinal cord slices and whole cell recording sites in models of neuropathic pain.

FIG. 2 shows samples of a continuous recording of excitatory postsynaptic currents evoked by stimulation of dorsal root afferents in the presence of vehicle alone (panel A; control) and in the presence of AOD9604 (also referred to herein as “LAT8881”; SEQ ID NO:2; panels B and C) for a period of 8-30 minutes. The recordings taken in the presence and absence of AOD9604 are superimposed in panel D.

FIG. 3 shows the effect of LAT8881 (SEQ ID NO:2) on spontaneous activity of WDR neurons in CCI rats. The results are shown as a percentage (%) of the control values. *p<0.05, compared to vehicle group at the same time points; #p<0.05, compared to the control; n=7 for each group.

FIG. 4 shows the effect of LAT8881 on wind-up of WDR neurons in CCI rats. The results are shown as a percentage (%) of the control values. *p<0.05, compared to vehicle group at the same time points, one-way ANOVA; #p<0.05, ##p<0.01, ###p<0.001, compared to the control, paired Student t-test; n=7 for each group.

FIG. 5 shows the effect of LAT8881 on DRG-generated ectopic discharge in CCI rats. The results are shown as a percentage (%) of the control values, unpaired t-test, ##p<0.01 compared to control, paired t-test; n=6 for vehicle and LAT8881 (1 mg/kg body weight, i.v.); n=5 for lidocaine (5 mg/kg body weight, i.v.).

FIG. 6 shows the effect of AOD9604 (10 μM) on postsynaptic properties of dorsal horn neurons. Samples of continuous recordings are provided, showing superimposed membrane responses to current injection in the absence (Control) or presence of AOD9604. The right hand side panel shows a plot of the data from the recordings shown on the left hand side. Note the decreased slope and intersection of the plots around −90 mV, consistent with the activation of potassium conductance.

FIG. 7 shows the effect of LAT8881 (1 mg/kg body weight, intravenously) on ectopic discharge of neuroma-origin in CCI rats, when compared to Vehicle (Control) and lidocaine. The results are shown as a percentage (%) of the control values; ###p<0.001 compared to control; n=6.

FIG. 8 shows the effect of LAT8881 (mg/kg body weight, intravenously) on ectopic discharge of DRG-origin in CCI rats, when compared to Vehicle (Control) and lidocaine. The results are shown as a percentage (%) of the control values; #p<0.05, ##p<0.01 compared to control; n=6; * p<0.05, compared to lidocaine; n=5.

FIG. 9 shows the effect of LAT8881 (intramuscularly (i.m.) administered at 0.1 mg/kg body weight, 0.5 mg/kg body weight, 1 mg/kg body weight, and 5 mg/kg body weight) on paw withdrawal threshold of the ipsilateral paws of Chung model rats when compared to Vehicle controls. Pre: pre-surgery; BL1: day 6-8 post-surgery; BL2: day 12-14 post-surgery; pre-dosing control; Vehicle: 1% DMSO in phosphate buffered saline (PBS) at 1 mL/kg body weight i.m.; *p<0.05, **p<0.01, *p<0.001 compared to the same time points in the Vehicle control animals (one-way ANOVA); n=8 for each group.

FIG. 10 shows the effect of LAT9991 (SEQ ID NO:4) intramuscularly administered at 0.5 mg/kg body weight, 1 mg/kg body weight, and 5 mg/kg body weight) on paw withdrawal threshold of the ipsilateral paws of Chung model rats when compared to Vehicle controls; Pre: pre-surgery; BL1: day 6-8 post-surgery; BL2: day 12-14 post-surgery; pre-dosing control; Vehicle: 1% DMSO in PBS (1 mL/kg body weight i.m.); Gabapentin (100 mg/kg body weight; i.m.); *p<0.05, **p<0.01, *p<0.001 compared to the same time points in the Vehicle control animals (one-way ANOVA); n=8 for each group.

FIG. 11 shows the effect of LAT8881 (administered by oral gavage at 1 mg/kg body weight, 2 mg/kg body weight, and 5 mg/kg body weight, PO) on paw withdrawal threshold of the ipsilateral paws of Chung model rats when compared to Vehicle controls; D-1 and D-2: pre-surgery; DO: day of surgery; D7: day 7 post-surgery; D14: day 14 post-surgery and time of dosing; Vehicle: 1% DMSO in PBS (2 mL/kg body weight PO); Gabapentin (100 mg/kg body weight; PO); **p<0.01 and *p<0.001 compared to the same time points in the Vehicle control animals (one-way ANOVA); n=8 for each group.

FIG. 12 shows the effect of LAT9991F (SEQ ID NO:5) administered by oral gavage at 1 mg/kg body weight, 2 mg/kg body weight, and 5 mg/kg body weight, PO) on paw withdrawal threshold of the ipsilateral paws of Chung model rats when compared to Vehicle controls; D-1 and D-2: 1 and 2 days pre-surgery; DO: day of surgery; D7: day 7 post-surgery; D14: day 14 post-surgery and time of dosing; Vehicle: 1% DMSO in PBS (2 mL/kg body weight PO); Gabapentin (100 mg/kg body weight; PO); *p<0.05, **p<0.01 and ***p<0.001 compared to the same time points in the Vehicle control animals (one-way ANOVA); n=8 for each group.

FIG. 13 shows the effect of LAT8881 (administered by oral gavage at 5 mg/kg body weight, PO) on paw withdrawal threshold (PWT) in an animal model of streptozotocin-induced diabetic neuropathy; Pre1, Pre2 and Pre3: baseline PWT readings at 1, 2 and 3 weeks before administration of streptozotocin (STZ); W1, W2: 1 and 2 weeks after STZ administration; D0-0 h: time of administration of LAT8881; D0-1 h, D0-2 h and D0-4 h: 1, 2 and 4 hours after STZ administration **p<0.01 compared to D0-0 h; paired Student t-test; n=6 for each group.

FIG. 14 shows the effect of LAT8881 (administered by oral gavage at 5 mg/kg body weight, PO) on paw withdrawal threshold (PWT) in an animal model of oxaliplatin-induced neuropathy; Pre1, Pre2 and Pre3: baseline PWT readings at 1, 2 and 3 weeks before administration of oxaliplatin; W1, W2: 1 and 2 weeks after oxaliplatin administration; D0-0 h: time of administration of LAT8881; D0-1 h, D0-2 h and D0-4 h: 1, 2 and 4 hours after oxaliplatin administration; **p<0.01 and ***p<0.001 compared to D0-0 h; paired Student t-test; n=6 for each group.

FIG. 15 shows the effect of LAT8881 (administered by oral gavage at 5 mg/kg body weight, PO) on paw withdrawal threshold (PWT) in an animal model of reserpine-induced fibromyalgia, a model of neuropathic pain; Pre1 and Pre2: baseline PWT readings at 1 and 2 weeks before administration of reserpine; DO: day of administration of reserpine; D8, D9 and D10: day 8, 9 and 10 after the first administration of reserpine; Dosing with Vehicle (5% DMSO in PBS, 2 mL/kg body weight, PO) and LAT8881 was at day 10 after the administration of reserpine (D8-0 h); *p<0.05 and **p<0.01 compared to Vehicle group; paired Student t-test; n=6 for LAT8881 group and n=3 for Vehicle group.

FIG. 16 shows the effect of LAT8881 (administered by oral gavage at 1 mg/kg body weight, PO, 5 mg/kg body weight and PO and 10 mg/kg body weight, PO) on paw withdrawal threshold (PWT) in an animal model of complete Freund's adjuvant (CFA)-induced inflammation (ipsilateral side), a model of nociceptive pain; D-2 and D-1: baseline PWT readings at 1 and 2 weeks before administration of CFA; D0: baseline reading on the day of administration of CPA; Baseline: reading taken on the day of administration of LAT8881; 1 hr, 2 hr and 4 hr: first, second and fourth hours after the administration of CFA; Dosing with Vehicle (5% DMSO in PBS, 2 mLkg body weight, PO), LAT8881 and Morphine (3 mg/kg body weight) was at day 1 after the administration of CFA; *p<0.05 and ***p<0.001 compared to Vehicle group at the same time points; one-way ANOVA; n=8 for each group.

FIG. 17 shows the detection of LAT8881 and LAT9991F in LAT8881-spiked human blood incubated at 37° C. over a 1 hour period. The results are shown as the peak area ratio of LAT8881 and LAT9991F to their respective internal standard values.

FIG. 18 shows the detection of LAT8881 and LAT9991F in LAT8881-spiked rat blood incubated at 37° C. over a 1 hour period. The results are shown as the peak area ratio of LAT8881 and LAT9991F to their respective internal standard values.

FIG. 19 shows the effect of LAT9991 (SEQ ID NO:4) administered by oral gavage at 1 mg/kg body weight, 2 mg/kg body weight, and 5 mg/kg body weight, PO) on paw withdrawal threshold of the ipsilateral paws of Chung model rats when compared to Vehicle controls; D-1 and D-2: 1 and 2 days pre-surgery; D0: day of surgery; D7: day 7 post-surgery; D14: day 14 post-surgery and time of dosing; Vehicle: 1% DMSO in PBS (2 mL/kg body weight PO); Gabapentin (100 mg/kg body weight; PO); *p<0.05, **p<0.01 and ***p<0.001 compared to the same time points in the Vehicle control animals (one-way ANOVA); n=8 for each group.

FIG. 20 shows spontaneous electrical activity in nerve cells in spinal cord slices from Chung model rats (upward deflections in the record), a characteristic feature of neuropathic pain states. Within 2 minutes of addition of 1 μM LATc9991F to the spinal cord slice, this electrical activity was suppressed; * indicates time that LATc9991F was added to the spinal cord slice preparation.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

### Methods of Treatment

The present invention is predicated, at least in part, on the inventors' surprising finding that peptides of formula (I) (SEQ ID NO:1) have advantageous analgesic properties, in that they are capable of alleviating neuropathic pain whilst having little or no analgesic effect on nociceptive pain. Thus, in an aspect disclosed herein, there is provided a method of treating neuropathic pain in a subject, the method comprising administering to a subject a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In a preferred embodiment, the peptide is YLRIVQCRSVEGSCGF (SEQ ID NO:2). SEQ ID NO:2 (referred to interchangeably herein as LAT8881 or AOD9604) is the C-terminal fragment of human growth hormone (hGH) spanning amino acid residues 178-192 of hGH (see, e.g., GenBank Accession numbers AAA72260.1, AML27053.1 and ADE06645.1), with an additional tyrosine residue at the N-terminus of the peptide.

The present inventors have also surprisingly found that the therapeutic effect on neuropathic pain is retained in the smaller fragments of the peptide of SEQ ID NO:2 For example, the inventors have surprisingly found that SEQ ID NO:4 (CRSVEGSCG; also referred to herein as LAT9991) has a therapeutically effective analgesic effect on neuropathic pain in vivo. Further, the inventors have surprisingly found that SEQ ID NO:5 (CRSVEGSCGF; also referred to herein as LAT9991F) also has a therapeutically effective analgesic effect on neuropathic pain in vivo. Thus, in an embodiment disclosed herein, R¹ is absent. In another embodiment, R² is absent. In yet another embodiment, R¹ and R² are absent.

In an embodiment disclosed herein, the peptide of formula (I) is from 9 to 16 amino acid residues in length, preferably 9, 10, 11, 12, 13, 14, 15 or 16 amino acid residues in length. The peptide of formula (I) will typically comprise a disulphide bond between the two cysteine (C) residues, thereby forming a cyclic peptide between the two cysteine residues.

In an embodiment, the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In a preferred embodiment, the peptide is CRSVEGSCG (SEQ ID NO:4). In another preferred embodiment, the peptide is CRSVEGSCGF (SEQ ID NO:5).

The present inventors have also surprisingly found that non-human variants of the peptides of formula (I) have similar analgesic properties to their human counterparts. Suitable non-human variants of the peptides of formula (I) will be familiar to persons skilled in the art, illustrative examples of which are disclosed in WO 2013/082667, the contents of which is incorporated herein by reference. In an aspect disclosed herein, there is provided a method of treating neuropathic pain in a subject, the method comprising administering to a subject a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

The peptide of formula (II) is from 9 to 17 amino acid residues in length, preferably 9, 10, 11, 12, 13, 14, 15, 16 or 17 amino acid residues in length. The peptide of formula (II) will typically comprise a disulphide bond between the two cysteine (C) residues, thereby forming a cyclic peptide between the two cysteine residues. In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In another embodiment, the peptide is CRRFVESSCAF (SEQ ID NO:9). In another embodiment, the peptide is CRRFVESSCA (SEQ ID NO:10).

In an embodiment, the peptide is formed as a pharmaceutically acceptable salt. It is to be understood that non-pharmaceutically acceptable salts are also envisaged, since these may be useful as intermediates in the preparation of pharmaceutically acceptable salts or may be useful during storage or transport. Suitable pharmaceutically acceptable salts will be familiar to persons skilled in the art, illustrative examples of which include salts of pharmaceutically acceptable inorganic acids, such as hydrochloric, sulphuric, phosphoric, nitric, carbonic, boric, sulfamic, and hydrobromic acids, or salts of pharmaceutically acceptable organic acids, such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids. Illustrative examples of suitable base salts include those formed with pharmaceutically acceptable cations, such as sodium, potassium, lithium, calcium, magnesium, ammonium and alkylammonium. Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

Also disclosed herein are prodrugs comprising the peptides of formulae (I) or (II), or the pharmaceutically acceptable salts thereof. As used herein, a “prodrug” typically refers to a compound that can be metabolized in vivo to provide the active peptide of formulae (I) or (II), or pharmaceutically acceptable salts thereof. In some embodiments, the prodrug itself also shares the same, or substantially the same, analgesic activity as the peptide of formulae (I) or (II), or pharmaceutically acceptable salts thereof, as described elsewhere herein.

In some embodiments, the peptides of formulae (I) or (II), or pharmaceutically acceptable salts thereof, may further comprise a C-terminal capping group. The term “C-terminal capping group”, as used herein, refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR^{b} where the C(O) is from the C-terminal carboxylic acid group and R^{a} is hydrogen, alkyl, alkenyl, alkynyl, cycloalkyl or aryl and R^{a} is alkyl, alkenyl, alkynyl, cycloalkyl or aryl. In particular embodiments, the C-terminal capping group is —NH₂, forming —C(O)NH₂. In some embodiments, the peptides of formulae (I) or (II), or pharmaceutically acceptable salts thereof, comprise a C-terminal polyethylene glycol (PEG). In an embodiment, the PEG has a molecular weight in the range of 220 to 5500 Da, preferably 220 to 2500 Da, more preferably 570 to 1100 Da.

In some embodiments, the peptides of formulae (I) or (II), or pharmaceutically acceptable salts thereof, may further comprise an N-terminal capping group. The term “N-terminal capping group”, as used herein, refers to a group that blocks the reactivity of the N-terminal amino group. Suitable N-terminal capping groups are acyl groups that form amide groups with the N-terminal amino group, for example, the N-terminal capping group forms a —NHC(O)R^{a} where the NH is from the N-terminal amino group and R^{a} is alkyl, alkenyl, alkynyl, cycloalkyl or aryl. In particular embodiments, the N-terminal capping group is —C(O)CH₃ (acyl), forming —NHC(O)CH₃.

In some embodiments, the peptides of formulae (I) or (II), or pharmaceutically acceptable salts thereof, may comprise a C-terminal capping group and an N-terminal capping group, as herein described.

The peptides of formulae (I) or (II), or pharmaceutically acceptable salts thereof, as herein described, can be made be any method known to persons skilled in the art. Illustrative examples of suitable methods include solution or solid phase synthesis using Fmoc or Boc protected amino acid residues, recombinant techniques using microbial culture, genetically engineered microbes, plants and recombinant DNA technology (see, e.g., Sambrook and Russell, Molecular Cloning: A Laboratory Manual (3^{rd} Edition), 2001, CSHL Press).

As described elsewhere herein, present inventors have surprisingly found, for the first time, that the peptides of formula (I) (SEQ ID NO:1) have advantageous analgesic properties, in that they are capable of alleviating neuropathic pain whilst having little or no analgesic effect on nociceptive pain. The peptides of formula (I) can therefore suitably be used to treat, prevent, alleviate or otherwise delay the onset of neuropathic pain in a subject, including one or more symptoms of neuropathic pain. The present inventors have also surprising found that non-human variants of the peptides of formula (I) have similar analgesic properties to their human counterparts.

The peptides of formulae (I) or (II), or pharmaceutically acceptable salts thereof, can therefore suitably be used to treat, prevent, alleviate or otherwise delay the onset of neuropathic pain in a subject, including one or more symptoms of neuropathic pain.

The terms “treating”, “treatment” and the like, are used interchangeably herein to mean relieving, reducing, alleviating, ameliorating or otherwise inhibiting neuropathic pain, including one or more symptoms of neuropathic pain, such as allodynia or hyperalgesia. The terms “treating”, “treatment” and the like are also used interchangeably herein to mean preventing the neuropathic pain from occurring or delaying the onset or subsequent progression of neuropathic pain in a subject that may be predisposed to, or at risk of, developing neuropathic pain, but has not yet been diagnosed as having it. In that context, the terms “treating”, “treatment” and the like are used interchangeably with terms such as “prophylaxis”, “prophylactic” and “preventative”.

The terms “treating”, “treatment” and the like also include relieving, preventing, reducing, alleviating, ameliorating or otherwise inhibiting the effects of the pain for at least a period of time. It is also to be understood that terms “treating”, “treatment” and the like do not imply that the neuropathic pain, or a symptom thereof, is permanently relieved, reduced, alleviated, ameliorated or otherwise inhibited and therefore also encompasses the temporary relief, reduction, alleviation, amelioration or otherwise inhibition of neuropathic pain, or a symptom thereof.

Without being bound by theory, or by a particular mode of application, neuropathic pain is typically characterised as pain which results from damage by injury or disease to nerve tissue or neurons per se or of dysfunction within nerve tissue. The pain may be peripheral, central or a combination thereof; in other words, the term “neuropathic pain” typically refers to any pain syndrome initiated or caused by a primary lesion or dysfunction in the peripheral or central nervous system. Neuropathic pain is also distinguishable in that it typically does not respond effectively to treatment by common pain medication such as opioids. By contrast, nociceptive pain is characterised as pain which results from stimulation of nociceptors by noxious or potentially harmful stimuli that may cause damage or injury to tissue. Nociceptive pain is typically responsive to common pain medication, such as opioids.

The term “analgesia” is used herein to describe states of reduced pain perception, including absence from pain sensations, as well as states of reduced or absent sensitivity to noxious stimuli. Such states of reduced or absent pain perception are typically induced by the administration of a pain-controlling agent or agents and occur without loss of consciousness, as is commonly understood in the art. Suitable methods for determining whether a compound is capable of providing an analgesic effect will be familiar to persons skilled in the art, illustrative examples of which include the use of animal models of neuropathic pain, such as chronic constriction injury, spinal nerve ligation and partial sciatic nerve ligation (see Bennett et al. (2003); Curr. Protoc. Neurosci., Chapter 9, Unit 9.14) and animal models of nociceptive pain, such as formalin-, carrageenan- or complete Freund's adjuvant (CFA)-induced inflammatory pain. Other suitable models of pain are discussed in Gregory et al. (2013, J. Pain.; 14(11); “An overview of animal models of pain: disease models and outcome measures”).

As persons skilled in the art will know, there are many possible causes of neuropathy and neuropathic pain. It is therefore to be understood that contemplated herein is the treatment or prevention of neuropathic pain regardless of cause. In some embodiments, neuropathic pain is a result of a disease or condition affecting the nerves (primary neuropathy) and/or neuropathy that is caused by systemic disease (secondary neuropathy), illustrative examples of which include diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathies; hereditary motor and sensory neuropathies (HMSN); hereditary sensory neuropathies (HSNs); hereditary sensory and autonomic neuropathies; hereditary neuropathies with ulcero-mutilation; nitrofurantoin neuropathy; tomaculous neuropathy; neuropathy caused by nutritional deficiency, neuropathy caused by kidney failure and complex regional pain syndrome. Other illustrative examples of conditions that may cause neuropathic pain include repetitive activities such as typing or working on an assembly line, medications known to cause peripheral neuropathy such as several antiretroviral drugs ddC (zalcitabine) and ddI (didanosine), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated with infectious processes (such as Guillain-Barré syndrome). Other illustrative examples of neuropathic pain include thermal or mechanical hyperalgesia, thermal or mechanical allodynia, diabetic pain, neuropathic pain affecting the oral cavity (e.g., trigeminal neuropathic pain, atypical odontalgia (phantom tooth pain), burning mouth syndrome), fibromyalgia and entrapment pain.

In an embodiment disclosed herein, the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy. peripheral neuropathy associated with infection, allodynia, hyperesthesia, hyperalgesia, burning pain and shooting pain.

In some embodiments, the neuropathic pain may be accompanied by numbness, weakness and loss of reflexes. The pain may be severe and disabling. By “hyperalgesia” is meant an increased response to a stimulus that is normally painful. A hyperalgesia condition is one that is associated with pain caused by a stimulus that is not normally painful. The term “hyperesthesia” refers to an excessive physical sensitivity, especially of the skin. The term “allodynia” as used herein refers to the pain that results from a non-noxious stimulus; that is, pain due to a stimulus that does not normally provoke pain. Illustrative examples of allodynia include thermal allodynia (pain due to a cold or hot stimulus), tactile allodynia (pain due to light pressure or touch), mechanical allodynia (pain due to heavy pressure or pinprick) and the like.

Neuropathic pain may be acute or chronic and, in this context, it is to be understood that the time course of a neuropathy may vary, based on its underlying cause. For instance, with trauma, the onset of neuropathic pain or symptoms of neuropathic pain may be acute, or sudden; however, the most severe symptoms may develop over time and persist for years. A chronic time course over weeks to months usually indicates a toxic or metabolic neuropathy. A chronic, slowly progressive neuropathy, such as occurs with painful diabetic neuropathy or with most hereditary neuropathies or with a condition termed chronic inflammatory demyelinating polyradiculoneuropathy (CIDP), may have a time course over many years. Neuropathic conditions with symptoms that relapse and remit include Guillain-Barre syndrome.

In some embodiments, neuropathic pain results from a condition characterised by neuronal hypersensitivity, such as fibromyalgia or irritable bowel syndrome.

In other embodiments, neuropathic pain results from a disorder associate with aberrant nerve regeneration resulting in neuronal hypersensitivity. Such disorders include breast pain, interstitial cystitis, vulvodynia and cancer chemotherapy-induced neuropathy.

In some embodiments, the neuropathic pain is related to surgery, pre-operative pain and post-operative pain, particularly post-operative neuropathic pain.

The term “subject”, as used herein, refers to a mammalian subject for whom treatment or prophylaxis of neuropathic pain is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In another embodiment, the subject is selected from the group consisting of a canine, a feline and an equine.

It is to be understood that a reference to a subject herein does not imply that the subject has neuropathic pain, or a symptom thereof, but also includes a subject that is at risk of developing neuropathic pain, or a symptom thereof. In an embodiment, the subject has (i.e., is experiencing) neuropathic pain or a symptom thereof. In another embodiment, the subject is not experiencing neuropathic pain or a symptom thereof at the time of treatment, but is at risk of developing neuropathic pain or a symptom thereof. In an illustrative example, the subject has a disease or condition that puts the subject at risk of developing neuropathic pain, for example, poorly managed diabetes, which may lead to a diabetic neuropathy. In another embodiment, the subject has had a disease or condition that has potential to result in neuropathic pain, such as herpes zoster (shingles), which may lead to post-herpetic neuralgia.

In some embodiments, the methods disclosed herein comprise administering a peptide of formula (II), or a pharmaceutically acceptable salt thereof, to a non-human subject. In a preferred embodiment, the non-human subject is selected from the group consisting of a canine, a feline or an equine. In other embodiments, the methods disclosed herein comprise administering a peptide of formula (I), or a pharmaceutically acceptable salt thereof, to a human subject. In other embodiments, the peptide of formula (I), or a pharmaceutically acceptable salt thereof, is administered to a non-human subject, such as a canine, a feline or an equine.

The peptides of formulae (I) or (II), or pharmaceutically acceptable salts thereof, are to be administered in a therapeutically effective amount. The phrase “therapeutically effective amount” typically means an amount necessary to attain the desired response, or to delay the onset or inhibit progression or halt altogether, the onset or progression of neuropathic pain being treated. It would be understood by persons skilled in the art that the therapeutically effective amount of peptide will vary depending upon several factors, illustrative examples of which include the health and physical condition of the subject to be treated, the taxonomic group of subject to be treated, the severity of the neuropathic pain to be treated, the formulation of the composition comprising a peptide of formula (I) or formula (II), or a pharmaceutically acceptable salt thereof, the route of administration, and combinations of any of the foregoing.

The therapeutically effective amount will typically fall within a relatively broad range that can be determined through routine trials by persons skilled in the art. Illustrative examples of a suitable therapeutically effective amount of the peptides of formulae (I) or (II), or pharmaceutically acceptable salts thereof, for administration to a human subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. In an embodiment disclosed herein, the therapeutically effective amount of the peptide of formula (I) or of formula (II), or pharmaceutically acceptable salts thereof, is from about 0.001 mg per kg of body weight to about 1 g per kg of body weight per dose (e.g., 0.001 mg/kg, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg of body weight, etc). In an embodiment, the therapeutically effective amount of the peptides of formulae (I) or (II), or the pharmaceutically acceptable salts thereof, is from about 0.001 mg to about 50 mg per kg of body weight. In an embodiment, the therapeutically effective amount of the peptides of formulae (I) or (II), or the pharmaceutically acceptable salts thereof, is from about 0.01 mg to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

As noted elsewhere herein, the present inventors have surprisingly found that a peptide of formula (I) possesses advantageous analgesic properties, whereby it is capable of alleviating neuropathic pain in a subject with minimal or no analgesic effect on nociceptive pain. Thus, in an embodiment disclosed herein, the peptide of formula (I), or the pharmaceutically acceptable salt thereof, is administered to the subject at a therapeutically effective amount that alleviates neuropathic pain in the subject in the absence of a therapeutically effective analgesic effect on nociceptive pain. The present inventors have also surprisingly found that non-human variants of the peptides of formula (I) have similar analgesic properties to their human counterparts. Thus, in an embodiment disclosed herein, the peptide of formula (II), or the pharmaceutically acceptable salt thereof, is administered to the subject at a therapeutically effective amount that alleviates neuropathic pain in the subject in the absence of a therapeutically effective analgesic effect on nociceptive pain.

By “therapeutically effective analgesic effect on nociceptive pain” is meant a reduction, either partial or complete, of a subject's perception of nociceptive pain. Thus, the absence of a therapeutically effective analgesic effect on nociceptive pain can be characterised, in an embodiment, by the subject retaining the ability to perceive a stimulus of nociceptive pain, to the same or substantially the same degree as if the subject had not received the peptide of formula (I) or of formula (II), or pharmaceutically acceptable salts thereof, despite a reduction of neuropathic pain. In an embodiment, the peptide of formula (I), or a pharmaceutically acceptable salt thereof, is not therapeutically effective for the treatment of nociceptive pain at a dosage suitable for treating neuropathic pain. In an embodiment, the peptide of formula (II), or a pharmaceutically acceptable salt thereof, is not therapeutically effective for the treatment of nociceptive pain at a dosage suitable for treating neuropathic pain.

In other embodiments, the peptide of formula (I), or a pharmaceutically acceptable salt thereof, is administered to the subject at a therapeutically effective amount that alleviates neuropathic pain in the subject with some, but otherwise a therapeutically ineffective, analgesic effect on nociceptive pain. In another embodiment, the peptide of formula (II), or a pharmaceutically acceptable salt thereof, is administered to the subject at a therapeutically effective amount that alleviates neuropathic pain in the subject with some, but otherwise a therapeutically ineffective, analgesic effect on nociceptive pain. The term “therapeutically ineffective analgesic effect on nociceptive pain” means there is either no discernible analgesic effect on nociceptive pain or a partial analgesic effect on nociceptive pain, but that the subject is still capable of perceiving a stimulus of nociceptive pain.

The peptide of SEQ ID NO:2 (YLRIVQCRSVEGSCGF; referred to interchangeably herein as AOD9604 or LAT8881) has previously been shown to be useful for the treatment of conditions such as obesity (see WO 99/12969 and WO 01/033977) and bone disorders (see WO 2005/105132). This peptide has also previously been shown to be useful for the treatment of inflammatory, traumatic or genetic diseases of muscle or connective tissue, attributed at least in part to increased production of chondrocytes, proteoglycan, collagen and cartilage tissue and to the promotion of muscle, ligament and tendon mass, form, repair and function (see WO 2013/082667). However, these earlier studies do not exemplify an analgesic effect on neuropathic pain.

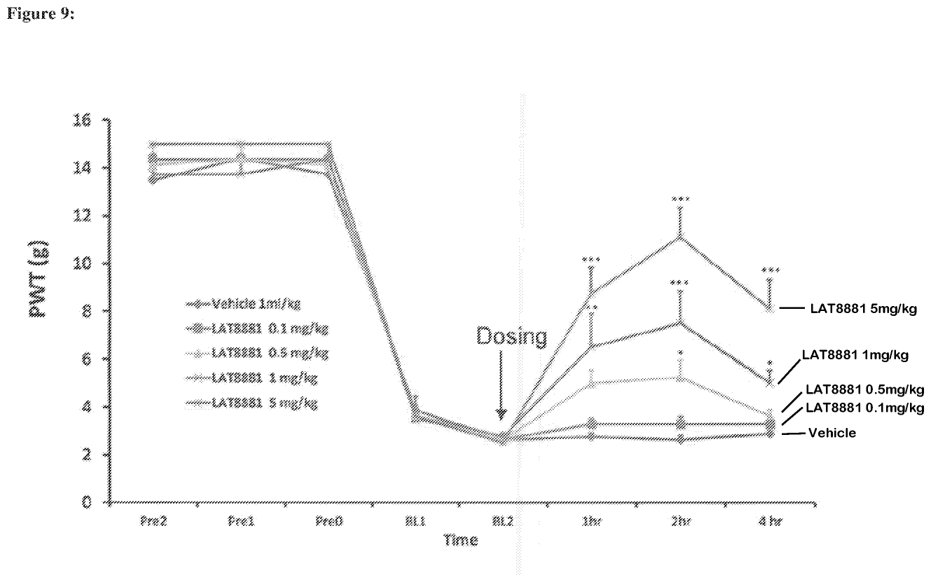

Cox et al. (2015; Drug Test. Analysis.; 7:31-38)) have previously reported on the breakdown products of AOD9604 (SEQ ID NO:2) in the presence of human serum and urine. The authors identified a single (shortest) metabolite, variously described throughout their paper as having the amino acid sequence of sometimes CRSVEGSCG or CRSVEGSCGF. From their report, it is unclear which metabolite is intended, noting that CRSVEGSCG is identified in Tables 1 and 4, whereas CRSVEGSCGF is identified at various passages in the text. In any event, the report by Cox et al. provides nothing whatsoever to suggest that this metabolite (whether CRSVEGSCG or CRSVEGSCGF) retains any biological activity, noting that the intent of the authors' study was to identify metabolites of AOD9604 for drug testing. The present inventor's have now shown, for the first time, that not only are these metabolites biologically active, but they retain an equivalent level of biological activity as compared to the parent peptide, SEQ ID NO: 2. This is evident, for example, from the data derived from the nerve constriction model of neuropathic pain (see FIGS. 9-16 and 19). As can be seen from that data, fragments of AOD9604 (SEQ ID NO:2) showed comparable activity to the parent peptide in terms of both magnitude and duration of neuropathic analgesia. Thus, in another aspect disclosed herein, there is provided a method of treating a condition in a subject, the method comprising administering to a subject a therapeutically effective amount of a peptide, or a pharmaceutically acceptable salt thereof, wherein the peptide consists, or consists essentially, of amino acid sequence CRSVEGSCG (SEQ ID NO:4) or amino acid sequence CRSVEGSCGF (SEQ ID NO:5), and wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, and a bone disorder.

### Routes of Administration

The peptides of formulae (I) and (II), and pharmaceutically acceptable salts thereof, may be administered to the subject by any suitable route that allows for delivery of the peptides to the subject at a therapeutically effective amount, as herein described. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation). The peptides of formulae (I) and (II), and pharmaceutically acceptable salts thereof, may also suitably be administered to the subject as a controlled release dosage form to provide a controlled release of the active agent(s) over an extended period of time. The term “controlled release” typically means the release of the active agent(s) to provide a constant, or substantially constant, concentration of the active agent in the subject over a period of time (e.g., about eight hours up to about 12 hours, up to about 14 hours, up to about 16 hours, up to about 18 hours, up to about 20 hours, up to a day, up to a week, up to a month, or more than a month). Controlled release of the active agent(s) can begin within a few minutes after administration or after expiration of a delay period (lag time) after administration, as may be required. Suitable controlled release dosage forms will be known to persons skilled in the art, illustrative examples of which are described in Anal, A. K. (2010; Controlled-Release Dosage Forms. Pharmaceutical Sciences Encyclopedia. 11:1-46).

Without being bound by theory or by a particular mode of application, it may be desirable to elect a route of administration on the basis of whether the neuropathic pain is localized or generalised. For example, where the neuropathic pain is localized, it may be desirable to administer the peptides to the affected area or to an area immediately adjacent thereto. For instance, where the neuropathic pain is in a joint (e.g., neck, knee, elbow, shoulder, hip, etc.), the peptides can be administered to the subject intra-articularly into the affected joint. Alternatively, or in addition, the peptides can be administered at, or substantially adjacent to, the affect joint. As another illustrative example, where the neuropathic pain is in the oral cavity (e.g., trigeminal neuropathic pain, atypical odontalgia (phantom tooth pain) or burning mouth syndrome), the peptides can be formulated for administration via the oral mucosa (e.g., by buccal and/or sublingual administration). Conversely, where the neuropathic pain is generalised or disseminated across multiple anatomical sites of a subject, the peptides may be administered topically, enterally and/or parenterally at any site with a view to distributing the active peptides across the multiple anatomical sites affected by neuropathic pain. In an embodiment disclosed herein, the peptides of formulae (I) or (II), or pharmaceutically acceptable salts thereof, are administered to the subject enterally. In an embodiment disclosed herein, the peptides of formulae (I) or (II), or pharmaceutically acceptable salts thereof, are administered to the subject orally. In an embodiment disclosed herein, the peptides of formulae (I) or (II), or pharmaceutically acceptable salts thereof, are administered to the subject parenterally. In another embodiment disclosed herein, the peptides of formulae (I) or (II), or pharmaceutically acceptable salts thereof, are administered to the subject topically. As described elsewhere herein, “topical” administration typically means application of the active agents to a surface of the body, such as the skin or mucous membranes, suitably in the form of a cream, lotion, foam, gel, ointment, nasal drop, eye drop, ear drop, transdermal patch, transdermal film (e.g., sublingual film) and the like. Topical administration also encompasses administration via the mucosal membrane of the respiratory tract by inhalation or insufflation. In an embodiment disclosed herein, the topical administration is selected from the group consisting of transdermal and transmucosal administration. In an embodiment, the peptides of formulae (I) or (II), or pharmaceutically acceptable salts thereof, are administered to the subject transdermally.

In an embodiment, the methods comprise orally administering the peptide of formula (I), or a pharmaceutically acceptable salt thereof, to a human. In another embodiment, the methods comprise orally administering the peptide of formula (I), or pharmaceutically acceptable salts thereof, to a non-human. subject. In yet another embodiment, the methods comprise orally administering the peptide of formula (I), or a pharmaceutically acceptable salt thereof, to a non-human subject selected from the group consisting of a feline, a canine and an equine.

In an embodiment, the methods comprise orally administering the peptide of formula (II), or a pharmaceutically acceptable salt thereof, to a human. In another embodiment, the methods comprise orally administering the peptide of formula (II), or a pharmaceutically acceptable salt thereof, to a non-human. subject. In yet another embodiment, the methods comprise orally administering the peptide of formula (II), or a pharmaceutically acceptable salt thereof, to a non-human subject selected from the group consisting of a feline, a canine and an equine.

In an embodiment, the methods comprise administering the peptide of formula (I), or a pharmaceutically acceptable salt thereof, topically to a human. In another embodiment, the methods comprise administering the peptide of formula (I), or a pharmaceutically acceptable salt thereof, topically to a non-human. subject. In yet another embodiment, the methods comprise administering the peptide of formula (I), or a pharmaceutically acceptable salt thereof, topically to a non-human subject selected from the group consisting of a feline, a canine and an equine.

In an embodiment, the methods comprise administering the peptide of formula (II), or a pharmaceutically acceptable salt thereof, topically to a human. In another embodiment, the methods comprise administering the peptide of formula (II), or a pharmaceutically acceptable salt thereof, topically to a non-human. subject. In yet another embodiment, the methods comprise administering the peptide of formula (II), or a pharmaceutically acceptable salt thereof, topically to a non-human subject selected from the group consisting of a feline, a canine and an equine.

In another embodiment, the methods comprise administering the peptide of SEQ ID NO:2, or a pharmaceutically acceptable salt thereof, orally to a human. In another embodiment, the methods comprise administering the peptide of SEQ ID NO:2, or pharmaceutically acceptable salts thereof, orally to a non-human. subject. In yet another embodiment, the methods comprise administering the peptide of SEQ ID NO:2, or pharmaceutically acceptable salts thereof, orally to a non-human subject selected from the group consisting of a feline, a canine and an equine.

In another embodiment, the methods comprise administering the peptide of SEQ ID NO:2, or a pharmaceutically acceptable salt thereof, topically to a human. In another embodiment, the methods comprise administering the peptide of SEQ ID NO:2, or pharmaceutically acceptable salts thereof, topically to a non-human. subject. In yet another embodiment, the methods comprise administering the peptide of SEQ ID NO:2, or pharmaceutically acceptable salts thereof, topically to a non-human subject selected from the group consisting of a feline, a canine and an equine.

In another embodiment, the methods comprise administering the peptide of SEQ ID NO:7, or pharmaceutically acceptable salts thereof, orally to a non-human. subject. In yet another embodiment, the methods comprise administering the peptide of SEQ ID NO:7, or pharmaceutically acceptable salts thereof, orally to a non-human subject selected from the group consisting of a feline, a canine and an equine.

In another embodiment, the methods comprise administering the peptide of SEQ ID NO:7, or pharmaceutically acceptable salts thereof, topically to a non-human. subject. In yet another embodiment, the methods comprise administering the peptide of SEQ ID NO:7, or pharmaceutically acceptable salts thereof, topically to a non-human subject selected from the group consisting of a feline, a canine and an equine.

Illustrative examples of topical administration are described elsewhere herein. In an embodiment, the topical administration is transdermal.

In an embodiment disclosed herein, the peptides of formulae (I) or (II), or pharmaceutically acceptable salts thereof, are administered to the subject as a controlled release dosage form, illustrative examples of which are described elsewhere herein. In an embodiment, the methods comprise administering the peptide of formula (I), or a pharmaceutically acceptable salt thereof, to a human as a controlled release dosage form. In another embodiment, the methods comprise administering the peptide of formula (I), or pharmaceutically acceptable salts thereof, to a non-human subject as a controlled release dosage form. In yet another embodiment, the methods comprise administering the peptide of formula (I), or a pharmaceutically acceptable salt thereof, as a controlled release dosage form to a non-human subject selected from the group consisting of a feline, a canine and an equine.

In another embodiment, the methods comprise administering the peptide of formula (II), or a pharmaceutically acceptable salt thereof, to a human as a controlled release dosage form. In another embodiment, the methods comprise administering the peptide of formula (II), or a pharmaceutically acceptable salt thereof, to a non-human subject as a controlled release dosage form. In yet another embodiment, the methods comprise administering the peptide of formula (II), or a pharmaceutically acceptable salt thereof, as a controlled release dosage form to a non-human subject selected from the group consisting of a feline, a canine and an equine.

In another embodiment, the methods comprise administering the peptide of SEQ ID NO:2, or a pharmaceutically acceptable salt thereof, to a human as a controlled release dosage form. In another embodiment, the methods comprise administering the peptide of SEQ ID NO:2, or pharmaceutically acceptable salts thereof, to a non-human subject as a controlled release dosage form. In yet another embodiment, the methods comprise administering the peptide of SEQ ID NO:2, or pharmaceutically acceptable salts thereof, as a controlled release dosage form to a non-human subject selected from the group consisting of a feline, a canine and an equine.

In another embodiment, the methods comprise administering the peptide of SEQ ID NO:7, or pharmaceutically acceptable salts thereof, to a non-human subject as a controlled release dosage form. In yet another embodiment, the methods comprise administering the peptide of SEQ ID NO:7, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, it may be beneficial to administer the peptides, as herein disclosed, via more than one route. For example, it may be desirable to administer a first dose parenterally (e.g., via intramuscular, intravenous; subcutaneous, epidural, intra-articular, intraperitoneal, intracisternal or intrathecal routes of administration) to induce a rapid or otherwise acute analgesic effect in a subject, followed by a subsequent (e.g., second, third, fourth, fifth, etc) dose administered enterally (e.g., orally or rectally) and/or topically (e.g., via transdermal or transmucosal routes of administration) to provide continuing availability of the active agent over an extended period subsequent to the acute phase of treatment. Alternatively, it may be desirable to administer a dose enterally (e.g., orally or rectally), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or topically (e.g., via transdermal or transmucosal routes of administration). Alternatively, it may be desirable to administer a dose topically (e.g., via transdermal or transmucosal routes of administration), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or enterally (e.g., orally or rectally).

The route of administration may suitably be selected on the basis of whether the neuropathic pain is localised or generalised, as discussed elsewhere herein. Alternatively, or in addition, the route of administration may suitably be selected having regard to factors such as the subject's general health, age, weight and tolerance (or a lack thereof) for given routes of administration (e.g., where there is a phobia of needles, an alternative route of administration may be selected, such as enteral and/or topical).

It is also to understood that, where multiple routes of administration are desired, any combination of two or more routes of administration may be used in accordance with the methods disclosed herein. Illustrative examples of suitable combinations include, but are not limited to, (in order of administration), (a) parenteral-enteral; (b) parenteral-topical; (c) parenteral-enteral-topical; (d) parenteral-topical-enteral; (e) enteral-parenteral; (f) enteral-topical; (g) enteral-topical-parenteral; (h) enteral-parenteral-topical; (i) topical-parenteral; (j) topical-enteral; (k) topical-parenteral-enteral; (1) topical-enteral-parenteral; (m) parenteral-enteral-topical-parenteral; (n) parenteral-enteral-topical-enteral; etc.

In an embodiment, the methods comprise (i) parenterally administering to the subject the peptides or compositions, as disclosed herein, and (ii) non-parenterally (i.e, enterally or topically) administering to the subject the peptides or compositions, as disclosed herein, wherein the non-parenteral (enteral or topical) administration is subsequent to the parenteral administration. In an embodiment, the parental administration is selected from the group consisting of intramuscular, a subcutaneous and intravenous. In a further embodiment, the parental administration is subcutaneous. In an embodiment, the non-parental administration is oral.

In an embodiment, the methods disclosed herein comprise (i) parenterally administering to a human subject the peptide of formula (I), or a pharmaceutically acceptable salt thereof, and (ii) orally administering to the human subject the peptide of formula (I), or a pharmaceutically acceptable salt thereof, wherein the oral administration is subsequent to the parenteral administration. In another embodiment, the methods disclosed herein comprise (i) parenterally administering to a human subject the peptide of SEQ ID NO:2, or a pharmaceutically acceptable salt thereof, and (ii) orally administering to the human subject the peptide of SEQ ID NO:2, or a pharmaceutically acceptable salt thereof, wherein the oral administration is subsequent to the parenteral administration. In an embodiment, the parental administration is subcutaneous. In another embodiment, the parental administration is intrathecal.

In an embodiment, the methods disclosed herein comprise (i) parenterally administering to a non-human subject the peptide of formula (II), or a pharmaceutically acceptable salt thereof, and (ii) orally administering to the non-human subject the peptide of formula (II), or a pharmaceutically acceptable salt thereof, wherein the oral administration is subsequent to the parenteral administration. In a further embodiment, the methods disclosed herein comprise (i) parenterally administering to a non-human subject the peptide of SEQ ID NO:7, or a pharmaceutically acceptable salt thereof, and (ii) orally administering to the non-human subject the peptide of SEQ ID NO:7, or a pharmaceutically acceptable salt thereof, wherein the oral administration is subsequent to the parenteral administration. In an embodiment, the non-human subject is selected from the group consisting of a feline, a canine and an equine. In an embodiment, the parental administration is subcutaneous. In another embodiment, the parental administration is intrathecal.

In a further embodiment, the methods disclosed herein comprise (i) parenterally administering to a human subject the peptide of formula (I), or a pharmaceutically acceptable salt thereof, and (ii) topically administering to the human subject the peptide of formula (I), or a pharmaceutically acceptable salt thereof, wherein the topical administration is subsequent to the parenteral administration. In a further embodiment, the methods disclosed herein comprise (i) parenterally administering to a human subject the peptide of SEQ ID NO:2, or a pharmaceutically acceptable salt thereof, and (ii) topically administering to the human subject the peptide of SEQ ID NO:2, or a pharmaceutically acceptable salt thereof, wherein the topical administration is subsequent to the parenteral administration.

In a further embodiment, the methods disclosed herein comprise (i) parenterally administering to a non-human subject the peptide of formula (II), or a pharmaceutically acceptable salt thereof, and (ii) topically administering to the non-human subject the peptide of formula (II), or a pharmaceutically acceptable salt thereof, wherein the topical administration is subsequent to the parenteral administration. In a further embodiment, the methods disclosed herein comprise (i) parenterally administering to a non-human subject the peptide of SEQ ID NO:7, or a pharmaceutically acceptable salt thereof, and (ii) topically administering to the non-human subject the peptide of SEQ ID NO:7, or a pharmaceutically acceptable salt thereof, wherein the topical administration is subsequent to the parenteral administration.

In an embodiment, the non-human subject is selected from the group consisting of a feline, a canine and an equine. In an embodiment, the parenteral route of administration is subcutaneous. In another embodiment, the topical route of administration is transdermal. In another embodiment, the parenteral administration is subcutaneous and the topical administration is transdermal.

Alternatively, or in addition, the peptides and compositions as herein described may suitably be administered as a controlled release dosage form. Thus, in an embodiment, the methods comprise (i) parenterally administering to the subject the peptides or compositions, as disclosed herein, and (ii) administering to the subject the peptides or compositions, as disclosed herein, as a controlled release dosage form, wherein the controlled release dosage form is administered subsequent to the parenteral administration. In another embodiment, the methods comprise (i) non-parenterally (enterally or topically) administering to the subject the peptides or compositions, as disclosed herein, and (ii) administering to the subject the peptides or compositions, as disclosed herein, as a controlled release dosage form, wherein the controlled release dosage form is administered to the subject subsequent to the non-parenteral administration. In yet another embodiment, the methods comprise (i) enterally administering to the subject the peptides or compositions, as disclosed herein, and (ii) administering to the subject the peptides or compositions, as disclosed herein, as a controlled release dosage form, wherein the controlled release dosage form is administered to the subject subsequent to the enteral administration. In yet another embodiment, the methods comprise (i) topically administering to the subject the peptides or compositions, as disclosed herein, and (ii) administering to the subject the peptides or compositions, as disclosed herein, as a controlled release dosage form, wherein the controlled release dosage form is administered to the subject subsequent to the topical administration. In a preferred embodiment, the controlled release dosage form is formulated for parenteral administration.

### Adjunct Therapy

The peptides of formulae (I) or (II), or pharmaceutically acceptable salts thereof, may suitably be administered together, either sequentially or in combination (e.g., as an admixture), with one or more another active agents. It will be understood by persons skilled in the art that the nature of the other active agents will depend on the condition to be treated or prevented. For example, where the subject has cancer, the peptides of formulae (I) or (II), or pharmaceutically acceptable salts thereof, may be administered to the subject together, either sequentially or in combination (e.g., as an admixture), with one or more chemotherapeutic agents, illustrative examples of which will be familiar to persons skilled in the art. Combination treatments of this nature can be advantageous by alleviating the neuropathic pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide. Thus, in an embodiment, the methods disclosed herein further comprise administering to the subject a therapeutically effective amount of a chemotherapeutic agent.

The peptides of formulae (I) or (II), or pharmaceutically acceptable salts thereof, may also be suitably administered to the subject together, either sequentially or in combination (e.g., as an admixture), with one or more other analgesic agents capable of alleviating pain in the subject (i.e., other than the peptides of formulae (I) and (II) and pharmaceutically acceptable salts thereof). Suitable analgesic agents will be familiar to persons skilled in the art, illustrative examples of which include analgesic agents capable of alleviating nociceptive pain, agents capable of alleviating neuropathic pain, or any combination thereof. Thus, in an embodiment, the methods disclosed herein further comprise administering to the subject a therapeutically effective amount of a second agent capable of alleviating pain in the subject, wherein the second agent is not the peptide of formula (I), or a pharmaceutically acceptable salt thereof.

In another embodiment, the methods disclosed herein further comprise administering to the subject a therapeutically effective amount of a second agent capable of alleviating pain in the subject, wherein the second agent is not the peptide of formula (II) or a pharmaceutically acceptable salt thereof.

In an embodiment, the second agent is capable of alleviating nociceptive pain in the subject. In another embodiment, the second agent is capable of alleviating neuropathic pain in the subject.

Suitable agents capable of alleviating nociceptive pain will be familiar to persons skilled in the art, illustrative examples of which include opiates such as morphine, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the second agent capable of alleviating nociceptive pain is an opioid.

In other embodiments disclosed herein, the peptides of formulae (I) or (II), or pharmaceutically acceptable salts thereof, are administered together, either sequentially or in combination (e.g., as an admixture), with another therapy to treat or alleviate neuropathic pain or the underlying condition that is causing the neuropathic pain. In some instances, the amount of the second neuropathic analgesic agent may be reduced when administration is together with a peptide of formula (I) or of formula (II), or pharmaceutically acceptable salts thereof. Illustrative examples of suitable agents capable of treating neuropathic pain include duloxetine, pregabalin, gabapentin, phenytoin, melatonin, carbamazepine, levocarnitine, capsaicin, tricyclic antidepressants such as amitryptiline and sodium channel blockers such as lidocaine.

### Pharmaceutical Compositions

The peptides of formulae (I) or (II), or pharmaceutically acceptable salts thereof, may be formulated for administration to a subject as a neat chemical. However, in certain embodiments, it may be preferable to formulate the peptides of formulae (I) or (II), or pharmaceutically acceptable salts thereof, as a pharmaceutical composition, including veterinary compositions. Thus, in another aspect disclosed herein, there is provided a pharmaceutical composition comprising a peptide of formula (I), or a pharmaceutically acceptable salt thereof, as described herein, for use in the treatment of neuropathic pain in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5).

In an embodiment, the peptide of formula (I), or a pharmaceutically acceptable salt thereof, is present in a therapeutically effective amount that, when administered to a subject, alleviates neuropathic pain in the subject in the absence of a therapeutically effective analgesic effect on nociceptive pain, as described elsewhere herein.

In an embodiment, the composition further comprises a second agent capable of alleviating pain in the subject, wherein the second agent is not the peptide of formula (I) or a pharmaceutically acceptable salt thereof. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment the second agent is an opioid.

In another aspect disclosed herein, there is provided a use of a peptide of formula (I), or a pharmaceutically acceptable salt thereof, as described herein, in the manufacture of a medicament for the treatment of neuropathic pain in a subject:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent.

In an embodiment, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5). In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5).

In an embodiment, the peptide of formula (I), or the pharmaceutically acceptable salt thereof, is formulated for administration to the subject in a therapeutically effective amount that alleviates neuropathic pain in the subject in the absence of a therapeutically effective analgesic effect on nociceptive pain, as described elsewhere herein.

In an embodiment, the peptide is formulated for administration sequentially, or in combination, with a second agent capable of alleviating pain in the subject, wherein the second agent is not the peptide of formula (I) or a pharmaceutically acceptable salt thereof, as described elsewhere herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In another aspect disclosed herein, there is provided a pharmaceutical composition comprising a peptide of formula (II), or a pharmaceutically acceptable salt thereof, for use in the treatment of neuropathic pain in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10).

In an embodiment, the peptide, or the pharmaceutically acceptable salt thereof, is present in a therapeutically effective amount that, when administered to a subject, alleviates neuropathic pain in the subject in the absence of a therapeutically effective analgesic effect on nociceptive pain, as described elsewhere herein.

In an embodiment, the composition further comprises a second agent capable of alleviating pain in the subject, wherein the second agent is not the peptide of formula (I) or a pharmaceutically acceptable salt thereof. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In another aspect disclosed herein, there is provided a use of a peptide of formula (II), or a pharmaceutically acceptable salt thereof, in the manufacture of a medicament for the treatment of neuropathic pain in a subject:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10). In an embodiment, the peptide of formula (II), or the pharmaceutically acceptable salt thereof, is formulated for administration to the subject in a therapeutically effective amount that alleviates neuropathic pain in the subject in the absence of a therapeutically effective analgesic effect on nociceptive pain, as described elsewhere herein.

In an embodiment, the peptide is formulated for administration sequentially, or in combination, with a second agent capable of alleviating pain in the subject, wherein the second agent is not the peptide of formula (II) or a pharmaceutically acceptable salt thereof, as described elsewhere herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

As noted elsewhere herein, the peptides of formulae (I) or (II), or pharmaceutically acceptable salts thereof, may be administered together, either sequentially or in combination (e.g., as an admixture), with one or more another active agents that will likely depend on the condition to be treated. For example, where the subject has cancer, the compositions disclosed herein may be formulated for administration together, either sequentially or in combination (e.g., as an admixture), with one or more chemotherapeutic agents, illustrative examples of which will be familiar to persons skilled in the art. Combination treatments of this nature can be advantageous by alleviating the neuropathic pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide.

The compositions disclosed herein may also be suitably formulated for administration to the subject together, either sequentially or in combination (e.g., as an admixture), with one or more other analgesic agents capable of alleviating pain in the subject (i.e., other than the peptides of formulae (I) and (H) and pharmaceutically acceptable salts thereof), as described elsewhere herein. In an embodiment, the compositions disclosed herein further comprise a second agent capable of alleviating pain in the subject, wherein the second agent is not the peptide of formula (I), or a pharmaceutically acceptable salt thereof.

In another embodiment, the compositions disclosed herein further comprise a second agent capable of alleviating pain in the subject, wherein the second agent is not the peptide of formula (II) or a pharmaceutically acceptable salt thereof.

In an embodiment, the second agent is capable of alleviating nociceptive pain in the subject. In another embodiment, the second agent is capable of alleviating neuropathic pain in the subject.

Suitable agents capable of alleviating nociceptive pain will be familiar to persons skilled in the art, illustrative examples of which include opiates such as morphine, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the second agent capable of alleviating nociceptive pain is an opioid.

In other embodiments disclosed herein, the compositions disclosed herein are formulated for administration together, either sequentially or in combination (e.g., as an admixture), with another therapy to treat or alleviate neuropathic pain or the underlying condition that is causing the neuropathic pain. In some instances, the amount of the second neuropathic analgesic agent may be reduced when administration is together with a peptide of formula (I) or of formula (II), or pharmaceutically acceptable salts thereof. Illustrative examples of suitable agents capable of treating neuropathic pain include duloxetine, pregabalin, gabapentin, phenytoin, melatonin, carbamazepine, levocarnitine, capsaicin, tricyclic antidepressants such as amitryptiline and sodium channel blockers such as lidocaine.

In another aspect disclosed herein, there is provided a pharmaceutical composition comprising:

(i) a peptide of formula (I), or a pharmaceutically acceptable salt thereof, as described herein:

(I)
(SEQ ID NO: 1)
R¹-CRSVEGSCG-R²

wherein
R¹ is selected from the group consisting of YLRIVQ, LRIVQ, RIVQ, IVQ, VQ, and Q, or R¹ is absent; and
R² is F (phenylalanine), or R² is absent; and
(ii) a second agent capable of alleviating pain in the subject, wherein the second agent is not the peptide of formula (I), or a pharmaceutically acceptable salt thereof, as described herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In an embodiment disclosed herein, the peptide of formula (I), or a pharmaceutically acceptable salt thereof, is formulated as a composition comprising a pharmaceutically acceptable carrier, excipient or diluent. The carrier, excipient or diluent is generally considered “acceptable” where they are compatible with the other ingredients of the composition and give rise to little or no deleterious effects in the recipient.

In another aspect disclosed herein, there is provided an analgesic composition comprising a peptide of formula (I), or a pharmaceutically acceptable salt thereof, as described herein:

(I)
(SEQ ID NO: 1)
R¹-CRSVEGSCG-R²

wherein
R¹ is selected from the group consisting of YLRIVQ, LRIVQ, RIVQ, IVQ, VQ, and Q or R¹ is absent; and
R² is F (phenylalanine), or R² is absent.

In an embodiment, the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5). In an embodiment, the analgesic composition further comprises a second agent capable of alleviating pain in the subject, as described elsewhere herein, wherein the second agent is not the peptide of formula (I) or a pharmaceutically acceptable salt thereof. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In another aspect disclosed herein, there is provided a pharmaceutical composition comprising:

(i) a peptide of formula (II), or a pharmaceutically acceptable salt thereof, as described herein:

(II)
(SEQ ID NO: 6)
R¹-CRRFVESSC-R²

wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent; and
(ii) a second agent capable of alleviating pain in the subject, wherein the second agent is not the peptide of formula (II) or a pharmaceutically acceptable salt thereof, as described herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In an embodiment disclosed herein, the peptide of formula (II), or a pharmaceutically acceptable salt thereof, is formulated as a composition comprising a pharmaceutically acceptable carrier, excipient or diluent. The carrier, excipient or diluent is generally considered “acceptable” where they are compatible with the other ingredients of the composition and give rise to little or no deleterious effects in the recipient.

In another aspect disclosed herein, there is provided an analgesic composition comprising a peptide of formula (II), or a pharmaceutically acceptable salt thereof, as described herein:

(II)
(SEQ ID NO: 6)
R¹-CRRFVESSC-R²

wherein
R¹ is selected from the group consisting of YLRVMK, LRVMK, RVMK, VMK, MK, and K, or R¹ absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10). In an embodiment, the analgesic composition further comprises a second agent capable of alleviating pain in the subject, as described elsewhere herein, wherein the second agent is not the peptide of formula (II) or a pharmaceutically acceptable salt thereof. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In another aspect disclosed herein, there is provided a composition comprising a therapeutically effective amount of a peptide, or a pharmaceutically acceptable salt thereof, wherein the peptide consists, or consists essentially, of amino acid sequence CRSVEGSCG (SEQ ID NO:4) or amino acid sequence CRSVEGSCGF (SEQ ID NO:5).

In another aspect disclosed herein, there is provided a composition comprising a therapeutically effective amount of a peptide, or a pharmaceutically acceptable salt thereof, wherein the peptide consists, or consists essentially, of amino acid sequence CRRFVESSCAF (SEQ ID NO:9) or CRRFVESSCA (SEQ ID NO:10).

In an embodiment, the composition further comprises a pharmaceutically acceptable carrier, excipient or diluent, as described elsewhere herein. In an embodiment, the composition is formulated for oral administration.

Illustrative examples of suitable pharmaceutical formulations include those suitable for enteral or parenteral administration, illustrative examples of which are described elsewhere herein, including oral, rectal, buccal, sublingual, vaginal, nasal, topical (e.g., transdermal), intramuscular, subcutaneous, intravenous, epidural, intra-articular and intrathecal.

The peptides of formulae (I) or (II), or pharmaceutically acceptable salts thereof, may suitably be placed into the form of pharmaceutical compositions and unit dosages thereof to be employed as solids (e.g., tablets or filled capsules) or liquids (e.g., solutions, suspensions, emulsions, elixirs, or capsules filled with the same) for oral use, in the form of ointments, suppositories or enemas for rectal administration, in the form of sterile injectable solutions for parenteral use (e.g., intramuscular, subcutaneous, intravenous, epidural, intra-articular and intrathecal administration); or in the form of ointments, lotions, creams, gels, patches, sublingual strips or films, and the like for parenteral (e.g., topical, buccal, sublingual, vaginal) administration. In an embodiment, the peptides of formulae (I) or (II), or pharmaceutically acceptable salts thereof, are formulated for topical (e.g., transdermal) delivery. Suitable transdermal delivery systems will be familiar to persons skilled in the art, illustrative examples of which are described by Prausnitz and Langer (2008; Nature Biotechnol. 26(11):1261-1268), the contents of which are incorporated herein by reference. In another embodiment, the peptides of formulae (I) or (II), or pharmaceutically acceptable salts thereof, are formulated for sublingual or buccal delivery. Suitable sublingual and buccal delivery systems will be familiar to persons skilled in the art, illustrative examples of which include dissolvable strips or films, as described by Bala et al. (2013; Int. J. Pharm. Investig. 3(2):67-76), the contents of which are incorporated herein by reference.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The peptides of formulae (I) or (II), or pharmaceutically acceptable salts thereof, as described herein, can be formulated for administration in a wide variety of enteral, topical and/or parenteral dosage forms. Suitable dosage forms may comprise, as the active component, either a peptide of formula (I), a peptide of formula (II), pharmaceutically acceptable salts thereof, or combinations of any of the foregoing, as herein described.

As noted elsewhere herein, it may be desirable to elect a route of administration on the basis of whether the neuropathic pain is localized or generalised. For example, where the neuropathic pain is localized, it may be desirable to formulate the compositions disclosed herein for administration to the affected area or to an area immediately adjacent thereto. For instance, where the neuropathic pain is in a joint (e.g., neck, knee, elbow, shoulder or hip), the composition can be formulated for intra-articular administration into the affected joint. Alternatively, or in addition, the composition can be formulated for administration at, or substantially adjacent to, the affect joint. As another illustrative example, where the neuropathic pain is in the oral cavity (e.g., trigeminal neuropathic pain, atypical odontalgia (phantom tooth pain) or burning mouth syndrome), the composition can be formulated for administration via the oral mucosa (e.g., by buccal and/or sublingual administration).

Conversely, where the neuropathic pain is generalised or disseminated across multiple anatomical sites of a subject, it may be convenient to formulate the composition for enteral, topical and/or parenteral route of administration, as described elsewhere herein, with a view to distributing the active agents across the multiple anatomical sites affected by neuropathic pain.

In an embodiment, the composition is formulated for oral administration to a human. In another embodiment, the composition is formulated for oral administration to a non-human subject. In yet another embodiment, the composition is formulated for oral administration to a non-human subject selected from the group consisting of a feline, a canine and an equine.

In another embodiment, the composition is formulated for parenteral administration to a human. In another embodiment, the composition is formulated for parenteral administration to a non-human subject. In yet another embodiment, the composition is formulated for parenteral administration to a non-human subject selected from the group consisting of a feline, a canine and an equine. In an embodiment, the parenteral administration is subcutaneous administration.

In another embodiment, the composition is formulated for topical administration to a human. In another embodiment, the composition is formulated for topical administration to a non-human subject. In yet another embodiment, the composition is formulated for topical administration to a non-human subject selected from the group consisting of a feline, a canine and an equine. In an embodiment, the topical administration is transdermal.

In another embodiment, the composition is formulated as a controlled release dosage form to be administered to a human. In another embodiment, the composition is formulated as a controlled release dosage form to be administered to a non-human subject. In yet another embodiment, the composition is formulated as a controlled release dosage form to be administered to a non-human subject selected from the group consisting of a feline, a canine and an equine. Illustrative examples of suitable controlled release dosage forms are described elsewhere herein.

For preparing pharmaceutical compositions of the peptides of formulae (I) and (II), or pharmaceutically acceptable salts thereof, pharmaceutically acceptable carriers can be either solid or liquid. Illustrative examples of solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier may be a finely divided solid which is in a mixture with the finely divided active component. In tablets, the active component may be mixed with the carrier having the necessary binding capacity in suitable proportions and compacted in the shape and size desired.

In some embodiments, the powders and tablets contain from five or ten to about seventy percent of the active compound. Illustrative examples of suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material, providing a capsule in which the active component, with or without carriers, is surrounded by a carrier. Similarly, cachets and lozenges are also envisaged herein. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The peptides of formulae (I) and (II), or pharmaceutically acceptable salts thereof, as described herein, may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active compound(s) may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also contemplated herein are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the peptides of formulae (I) or (II), or pharmaceutically acceptable salts thereof, as described herein, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the peptides used in the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, or in addition, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the peptide will generally have a small particle size for example of the order of 1 to 10 microns or less. Such a particle size may be obtained by means known in the art, for example by micronization.

When desired, formulations adapted to give controlled or sustained release of the active ingredient may be employed, as described elsewhere herein.

In an embodiment, the pharmaceutical preparations, as herein described, are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In another aspect disclosed herein, there is provided a composition comprising a peptide of SEQ ID NO: 4 or SEQ ID NO:5, or a pharmaceutically acceptable salt thereof, as herein described, for use as a medicament.

In another aspect disclosed herein, there is provided a composition comprising a peptide of SEQ ID NO: 9 or SEQ ID NO:10, or a pharmaceutically acceptable salt thereof, as herein described, for use as a medicament.

In an embodiment, the compositions disclosed herein are formulated for oral administration to a human. In yet another embodiment, the compositions disclosed herein are formulated for oral administration to a non-human. In a further embodiment, the compositions disclosed herein are formulated for oral administration to a non-human selected from the group consisting of a feline, a canine and an equine.

In another embodiment, the peptide of formula (I), or a pharmaceutically acceptable salt thereof, as disclosed herein, is formulated for oral administration to a human. subject. In another embodiment, the peptide of formula (I), or a pharmaceutically acceptable salt thereof, as disclosed herein, is formulated for oral administration to a non-human. subject. In yet another embodiment, the peptide of formula (I), or a pharmaceutically acceptable salt thereof, as disclosed herein, is formulated for oral administration to a non-human subject selected from the group consisting of a feline, a canine and an equine.

In another embodiment, the peptide of formula (I), or a pharmaceutically acceptable salt thereof, as disclosed herein, is formulated for topical administration to a human. subject. In yet another embodiment, the peptide of formula (I), or a pharmaceutically acceptable salt thereof, as disclosed herein, is formulated for topical administration to a non-human subject. In another embodiment, the peptide of formula (I), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (I), or a pharmaceutically acceptable salt thereof, as disclosed herein, is formulated for administration to a human subject as a controlled release dosage form. In yet another embodiment, the peptide of formula (I), or a pharmaceutically acceptable salt thereof, as disclosed herein, is formulated for administration to a non-human subject as a controlled release dosage form. In another embodiment, the peptide of formula (I), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (II), or a pharmaceutically acceptable salt thereof, as disclosed herein, are formulated for oral administration to a non-human. subject. In yet another embodiment, the peptide of formula (II), or a pharmaceutically acceptable salt thereof, as disclosed herein, is formulated for oral administration to a non-human subject selected from the group consisting of a feline, a canine and an equine.

In another embodiment, the peptide of formula (II), or a pharmaceutically acceptable salt thereof, as disclosed herein, is formulated for topical administration to a non-human. subject. In yet another embodiment, the peptide of formula (II), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (II), or a pharmaceutically acceptable salt thereof, as disclosed herein, is formulated for administration to a human subject as a controlled release dosage form. In yet another embodiment, the peptide of formula (II), or a pharmaceutically acceptable salt thereof, as disclosed herein, is formulated for administration to a non-human subject as a controlled release dosage form. In another embodiment, the peptide of formula (II), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:2, or a pharmaceutically acceptable salt thereof, is formulated for oral administration to a human. In another embodiment, the peptide of SEQ ID NO:2, or a pharmaceutically acceptable salt thereof, is formulated for oral administration to a non-human. subject. In yet another embodiment, the peptide of SEQ ID NO:2, or a pharmaceutically acceptable salt thereof, is formulated for oral administration to a non-human subject selected from the group consisting of a feline, a canine and an equine.

In another embodiment, the peptide of SEQ ID NO:2, or a pharmaceutically acceptable salt thereof, as disclosed herein, is formulated for topical administration to a human. subject. In yet another embodiment, the peptide of SEQ ID NO:2, or a pharmaceutically acceptable salt thereof, as disclosed herein, is formulated for topical administration to a non-human subject. In another embodiment, the peptide of SEQ ID NO:2, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:2, or a pharmaceutically acceptable salt thereof, as disclosed herein, is formulated for administration to a human subject as a controlled release dosage form. In yet another embodiment, the peptide of SEQ ID NO:2, or a pharmaceutically acceptable salt thereof, as disclosed herein, is formulated for administration to a non-human subject as a controlled release dosage form. In another embodiment, the peptide of SEQ ID NO:2, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:7, or a pharmaceutically acceptable salt thereof, is formulated for oral administration to a non-human. subject. In yet another embodiment, the peptide of SEQ ID NO:7, or a pharmaceutically acceptable salt thereof, is formulated for oral administration to a non-human subject selected from the group consisting of a feline, a canine and an equine.

In another embodiment, the peptide of SEQ ID NO:7, or a pharmaceutically acceptable salt thereof, is formulated for topical administration to a non-human. subject. In yet another embodiment, the peptide of SEQ ID NO:7, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:7, or a pharmaceutically acceptable salt thereof, as disclosed herein, is formulated for administration to a human subject as a controlled release dosage form. In yet another embodiment, the peptide of SEQ ID NO:7, or a pharmaceutically acceptable salt thereof, as disclosed herein, is formulated for administration to a non-human subject as a controlled release dosage form. In another embodiment, the peptide of SEQ ID NO:7, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, the compositions disclosed herein can be suitably formulated for administration via said multiple routes. For example, it may be desirable to administer a first dose parenterally (e.g., intramuscular, intravenously; subcutaneously, etc) to induce a rapid or otherwise acute analgesic effect in a subject, followed by a subsequent (e.g., second, third, fourth, fifth, etc) dose administered non-parenterally (e.g., enterally and/or topically) to provide continuing availability of the active agent over an extended period subsequent to the acute phase of treatment. Thus, in an embodiment, the peptides and compositions, as disclosed herein, are formulated for parenteral administration to the subject as a first dose (i.e., as a parenteral dosage form) and formulated for non-parenteral administration to the subject after the first dose (e.g., as an enteral and/or topical dosage form). In an embodiment, the parental administration is selected from the group consisting of intramuscular, subcutaneous and intravenous. In a further embodiment, the parental administration is subcutaneous.

In another embodiment, the enteral administration is oral administration. Thus, in an embodiment, the peptides and compositions, as disclosed herein, are formulated for parenteral administration to the subject as a first dose and formulated for oral administration to the subject after the first dose (i.e., as an oral dosage form).

In another embodiment, the enteral administration is topical administration. Thus, in an embodiment, the peptides and compositions, as disclosed herein, are formulated for parenteral administration to the subject as a first dose and formulated for topical administration to the subject after the first dose (i.e., as an oral dosage form). In an embodiment, the topical administration is transdermal administration.

In another embodiment, it may be desirable to administer a first dose parenterally (e.g., intramuscular, intravenously; subcutaneously, etc) to induce a rapid or otherwise acute analgesic effect in a subject, followed by a subsequent (e.g., second, third, fourth, fifth, etc) administration of a controlled release dosage form, as described elsewhere herein, to provide a controlled release of the active agent over an extended period subsequent to the acute phase of treatment. Thus, in another embodiment, the peptides and compositions, as disclosed herein, are formulated for parenteral administration to the subject as a first dose and formulated as a controlled release dosage form to be administered to the subject after the first dose. In an embodiment, the controlled release dosage form is formulated for parental administration.

It may also be desirable to administer a first dose enterally (e.g., orally or rectally), followed by a subsequent (e.g., second, third, fourth, fifth, etc) dose administered topically (e.g., transdermally). Thus, in an embodiment, the peptides and compositions, as disclosed herein, are formulated for enteral administration to the subject as a first dose (i.e., as an enteral dosage form; oral or rectal) and formulated for topical administration to the subject after the first dose (e.g., as a transdermal or transmucosal dosage form). In another embodiment, the peptides and compositions, as disclosed herein, are formulated for topical administration selected from the group consisting of transdermal and transmucosal administration. In a further embodiment, the peptides and compositions, as disclosed herein, are formulated for transdermal administration.

In yet another embodiment, it may be desirable to administer the peptides or compositions, as disclosed herein, enterally (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for administration as a first dose enterally and formulated for administration as a controlled release dosage form, wherein the controlled release dosage form is formulated for administration subsequent to the first dose. In an embodiment, the enteral dose is formulated for oral administration. In another embodiment, the controlled release dosage form is formulated for parenteral administration.

In an embodiment, it may be desirable to administer the peptides or compositions, as disclosed herein, topically (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for topical administration as a first dose and formulated for administration as a controlled release dosage form, wherein the controlled release dosage form is formulated for administration subsequent to the first topical dose. In an embodiment, the topical dose is formulated for transdermal administration. In another embodiment, the controlled release dosage form is formulated for parenteral administration.

The invention will now be described with reference to the following Examples which illustrate some preferred aspects of the present invention. However, it is to be understood that the particularity of the following description of the invention is not to supersede the generality of the preceding description of the invention.

### EXAMPLES

Peptides comprising the amino acid sequence of SEQ ID NOs:2 and 5 were synthesized by Auspep (Victoria, Australia) using solid phase synthesis and Fmoc protection strategy.

### Example 1: In Vitro Electrophysiological Properties

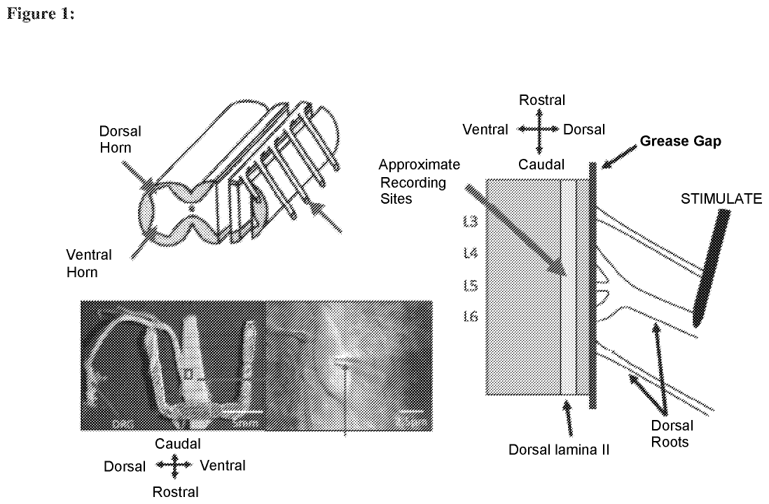

An in vitro spinal cord slice with intact dorsal root afferents combined with single-cell whole-cell patch clamp electrophysiological recording techniques was used to assess the electrophysiological properties of the peptide having the amino acid sequence of SEQ ID NO:2. A schematic diagram of the experimental preparation is shown in FIG. 1.

Spinal cord slices were prepared from chronic nerve constriction models of neuropathic pain (Chung models) and tested against the peptide of SEQ ID NO:2.

The spinal nerve ligation model (Chung model) was first reported by Kim and Chung (1992; Pain, 50(3):355-63) and involves a single tight ligation of the L5 spinal nerve. The model shows characteristic features of neuropathic pain symptoms/signs such as: mechanical allodynia, mechanical and thermal hyperalgesia and spontaneous pain that mimics the symptoms/signs observed in clinical patients. This model has been used as a “gold standard’ model for assessing the efficacy of novel compounds targeting neuropathic pain.

Adult male Sprague-Dawley rats, 8-9 weeks old, weighing 220-250 g at the time of surgery, were purchased from Charles River UK Ltd. The animals were housed in groups of 4 in an air-conditioned room on a 12-hour light/dark cycle. Food and water were available ad libitum. They were allowed to acclimatise to the experimental environment for three days by leaving them on a raised metal mesh for at least 40 min. The baseline paw withdrawal threshold (PWT) was examined using a series of graduated von Frey hairs for 3 consecutive days before surgery and re-assessed on the 6th to 8th day after surgery and on the 12th to 14th day after surgery before drug dosing Each rat was anaesthetized with 5% isoflurane mixed with oxygen (2 L per min) followed by an intramuscular (i.m.) injection of ketamine 90 mg/kg plus xylazine 10 mg/kg. The back was shaved and sterilized with povidone-iodine. The animal was placed in a prone position and a para-medial incision was made on the skin covering the L4-6 level. The L5 spinal nerve was carefully isolated and tightly ligated with 6/0 silk suture. The wound was then closed in layers after a complete hemostasis. A single dose of antibiotics (Amoxipen, 15 mg/rat, i.p.) was routinely given for prevention of infection after surgery. The animals were placed in a temperature-controlled recovery chamber until fully awake before being returned to their home cages

Chung model rats, aged 8 to 12 weeks, were housed in an air-conditioned room on a 12 hour light/dark cycle with food and water available ad libitum. The rats were terminally anaesthetized using isofluorane and decapitated. The vertebral column, rib cage and surrounding tissues were rapidly removed and pinned under ice-cold (<4° C.), high sucrose-containing artificial cerebrospinal fluid (aCSF) comprising: 127 mM sucrose, 1.9 mM KCl, 1.2 mM KH₂PO₄, 0.24 mM CaCl₂, 3.9 mM MgCl₂, 26 mM NaHCO₃, 10 mM D-glucose and 0.5 mM ascorbic acid. A laminectomy was performed and the spinal cord and associated roots gently dissected and teased out of the spinal column and surrounding tissues. Dura and pia mater and ventral roots were subsequently removed with fine forceps and the spinal cord hemisected. Care was taken to ensure dorsal root inputs to the spinal cord were maintained. The hemisected spinal cord-dorsal root preparations were secured to a tissue slicer and spinal cord slices (400 to 450 μm thick) with dorsal roots attached were cut in chilled (<4° C.) high sucrose aCSF using a Leica VT1000s microtome.

Slices were transferred to a small beaker containing ice cold aCSF with 127 mM NaCl, 1.9 mM KCl, 1.2 mM KH₂PO₄, 1.3 mM MgCl₂, 2.4 mM CaCl₂, 26 mM NaHCO₃ and 10 mM D-glucose, and rapidly warmed to 35° C. 1° C. in a temperature-controlled water bath over a 20 minute period, then subsequently removed and maintained at room temperature (22° C.±2° C.) prior to electrophysiological recording. Electrophysiological recording was performed in aCSF comprising 127 mM NaCl, 1.9 mM KCl, 1.2 mM KH₂PO₄, 1.3 mM MgCl₂, 2.4 mM CaCl₂, 26 mM NaHCO₃ and 10 mM D-glucose.

Whole-cell recordings were performed at 34-35° C. from Lamina I or II neurones in the dorsal horm of the spinal cord slices using Axopatch 1D and/or Multiclamp 700B amplifies and using the “blind” version of the patch-clamp technique.

Patch pipettes were pulled from thin-walled borosilicate glass with resistances of between 3 and 8 M when filled with intracellular solution. Biocytin was included in the patch solution to allow post-recording identification of the recorded neurones. The peptide of SEQ ID NO:2 (AOD; also referred to herein as LAT8881) was applied to the recorded tissue in the tissue bath at a concentration of 10 μM.

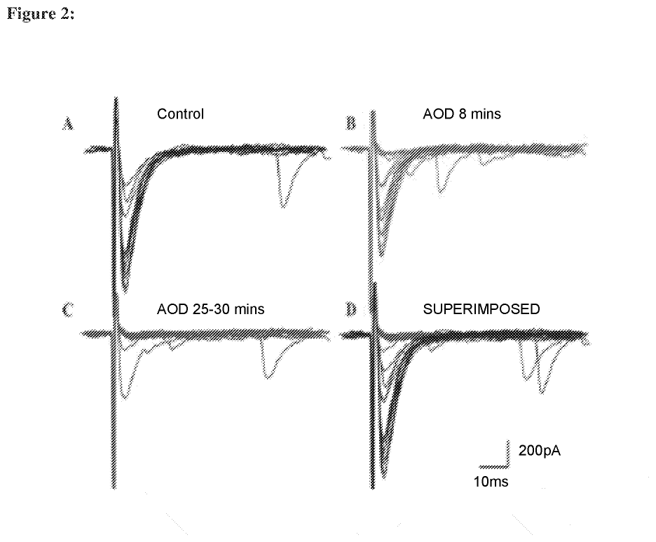

The effect of AOD9604 (SEQ ID NO:2) on post-synaptic current following stimulation of dorsal root afferents was detectable by 8 minutes and almost completely suppressed post-synaptic currents at 25 mins (see FIG. 2). By contrast, human growth hormone had no effect on post-synaptic current (data not shown).

The effect on post-synaptic current by AOD was at least partially reversible on washout, suggesting that AOD was not toxic to the nerve cells.

### Example 2: In Vivo Electrophysiological Properties

This study was undertaken to assess the effect of LAT8881 (SEQ ID NO:2) on spontaneous activity of WDR neurones in a chronic nerve constriction model using CCI rats, which were prepared as outlined above. Briefly, after behavioural validation, rats were anaesthetized with urethane 1.2 g/kg, i.p., for induction and subsequently topped up at 0.1-0.5 g/kg, i.v., for maintenance, if required. The right carotid artery and jugular vein were cannulated separately to monitor blood pressure and permit drug administration, respectively. Body temperature was monitored and controlled within a physiological range through a thermo-blanket system. Electrocardiogram (ECG) was routinely monitored through a pair of stainless steel needles inserted into the left and right forepaws.

For ectopic discharge of neuroma-origin, an incision was made on the lateral side of left hindlimb. Under a dissection microscope, the sural nerve below the sciatic nerve ligation area was exposed and carefully isolated from the surrounding connective tissues. The skin was stitched to a metal O-ring to form a pool which was later filled with warm mineral oil to protect the nerve. The sciatic nerve above the ligation area was sectioned The nerve sheath was then carefully removed. A small bundle of nerve filaments was teased from the distant cut end of the sural nerve and looped on to a unipolar silver wire recording electrode with a reference electrode connected to the connective tissues nearby.

For ectopic discharge of DRG-origin, the basic dissection and recording procedures were the same as recording set-up for that of neuroma-origin, with the only difference being the recording site of the sciatic nerve was above the ligation area, below the DRG

For recording WDR neurones from spinal cord dorsal horn, a laminectomy was carried out to expose the T11 to L2 segments. The incised back skin was clamped to a plastic film to form an oil pool to prevent the surface of the spinal cord from becoming dehydrated. The dura mater over the exposed spinal cord was opened. For dorsal horn neurone recording, carbon-fibre microelectrodes (Impedance, 0.4-0.8 MO at 1 KHz) were lowered into the spinal cord dorsal horn using a manual hydraulic manipulator to record neuronal activity. The neurones recorded were from deep layers (Lamina IV or V at about 500 to 900 μm from the surface of spinal cord) of the L4 to L5 level. The proprioceptive neurones innervating muscle spindles, joint receptors etc were excluded according to their firing pattern and responses to joint movement. The neural activity was amplified and monitored using standard electrophysiological recording techniques and recorded on to a PC using CED Spike 5 software (Cambridge Electronics Design, CED).

The electrical signal was amplified through a Digitimer AC amplifier (NL104) and filtered with a low-pass filter set at 50-500 Hz and high cut at 500 to 5 KHz. The signals were then recorded through a CED micro-1401 interface to a PC and analysed off-line.

### (i) Identification of WDR Neurones

Methods used to identify WDR neurones from the dorsal horn have been reported previously (Elmes et al., 2004). In brief, a set protocol of mechanical stimulation was used to identify WDR neurones after peripheral receptive field (RF) in the hind-paw was mapped out: firstly, gentle brush for 10 s; secondly, three different sizes of von Frey hair (1 g, 4 g, 15 g), applied to the RF with 1 s on and 1 s off and repeated 10 times (the interval between two von Frey Hair applications was 10 s); thirdly, a 10 s pinch-stimulus was applied using a pair of small forceps on the RF. The responses of a typical WDR neurone would increase as the intensity of stimulation increased. A. Brush (10 s)2 s B. von Frey Hair (1 s on-1 s off 10 times) 20 s 1 g 4 g 15 g C. Pinch (10 s)2 s D. Wind-up and after-discharge 3 s.

(ii) Induction of Wind-Up and after-Discharge

After a WDR neurone was identified, a pair of fine needle electrodes was inserted into the RF to deliver electrical stimulation. Thresholds for evoking action potentials of C-fibre responses (latency 90 ˜300 ms after electrical stimulation) were determined by delivering 1 ms duration single electrical pulses of increasing strength. Once the thresholds were found, a train of electrical pulses (16 pulses in 5 s, 1 ms duration) at an intensity of two times threshold was delivered, once every 5 minutes. The neural activity (spontaneous firing and evoked responses) was recorded for at least 20 minutes before the vehicle or compound administration and then for a further 40 min after the vehicle or compound was injected.

(iii) Measurement of Spontaneous Activity

The average spontaneous firing frequency over consecutive 4.5 min periods (expressed as numbers of action potentials per minute), immediately before electrical stimulation, was measured before and at 10, 20, 30, 40, 50, 60 min after vehicle or compound injection.

### (iv) Measurement of Wind-Up

Wind-up was measured using a protocol based on the methods described by Svendsen, et al., 1999. Briefly, wind-up was calculated as the total number of evoked action potentials of a neuron in response to all 16 electrical pulses minus 16 times the action potentials induced by the first electrical pulse in that train. Firstly, the number of action potentials within 300 ms after first electrical stimulation pulse (A) was counted. Secondly, the total number of action potentials induced by the whole train of electrical pulses (16 pulses) in that 5 s (B) were counted. Then the number of wind-up action potential was calculated as follows:

(v) Wind-up action potential number=B−(A×16)

The wind-up action potential numbers immediately before compound administration (0 min) and every 10 min after compound injection were counted. In some cases, wind-up was completely inhibited following compound administration, the number of wind-up action potentials (B) was even lower than control levels (A×16) leading to a negative reading. In such cases, the wind-up was set as 0 (completely inhibited) for ease of statistical analysis.

(vi) Measurement of after-Discharge:

The total number of action potentials recorded within 10 s starting from 300 ms after the last electrical pulse of the train (i.e. the 16th electrical stimulus) was used as a marker or indicator of the extent of after-discharge for that neuron.

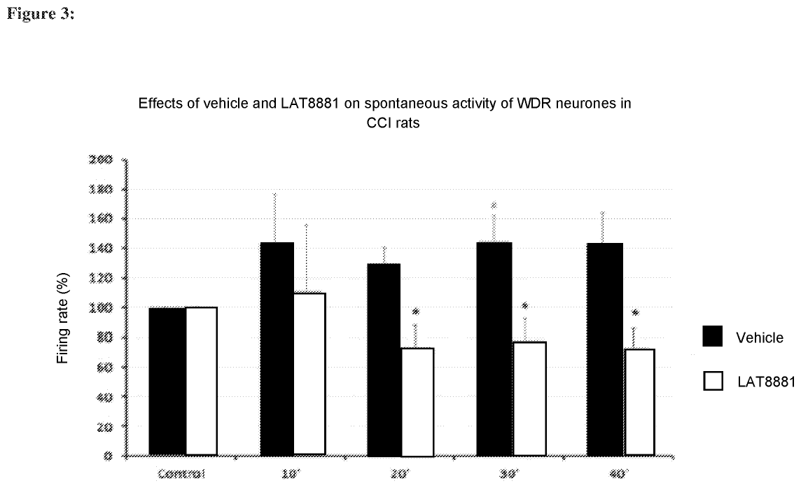

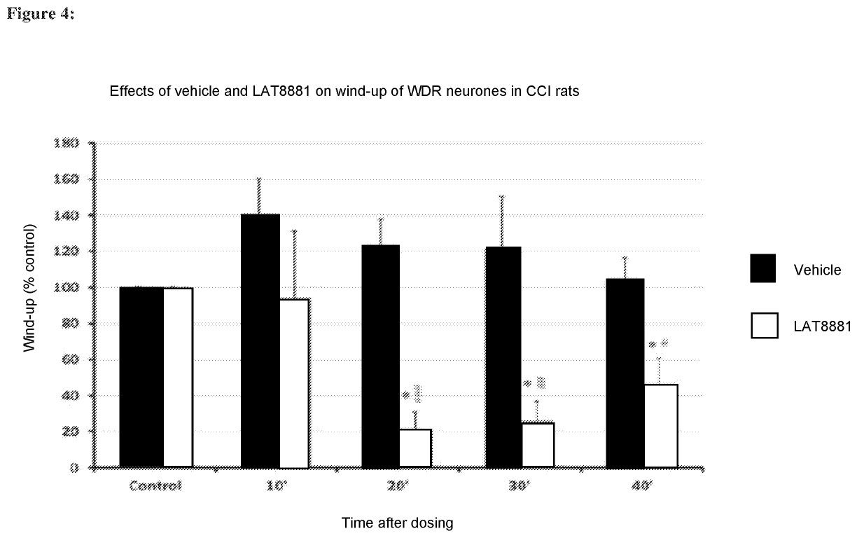

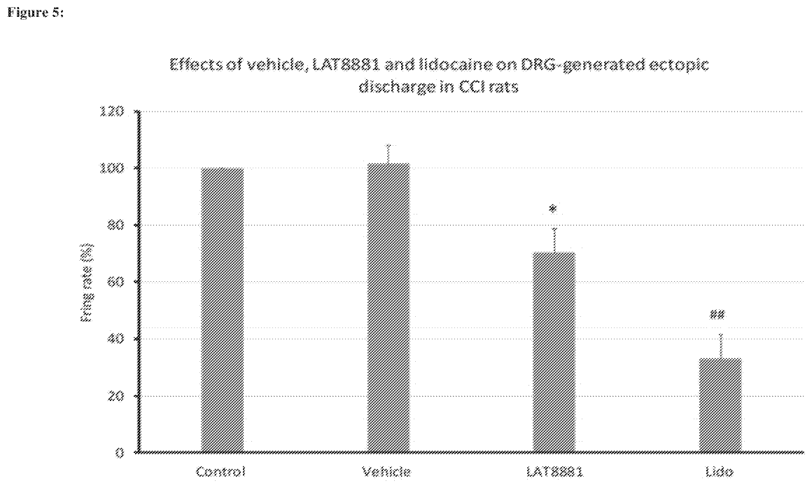

As shown in FIG. 3, LAT8881 suppressed spontaneous activity in WDR neurons in this chronic nerve constriction model. LAT8881 also suppressed wind-up on WDR neurons in this model (see FIG. 4). When administered intravenously, LAT8881 also suppressed the DRG-generated discharge in this chronic nerve constriction model when compared to vehicle (see FIG. 5).

### Example 3: Ex Vivo Electrophysiological Properties

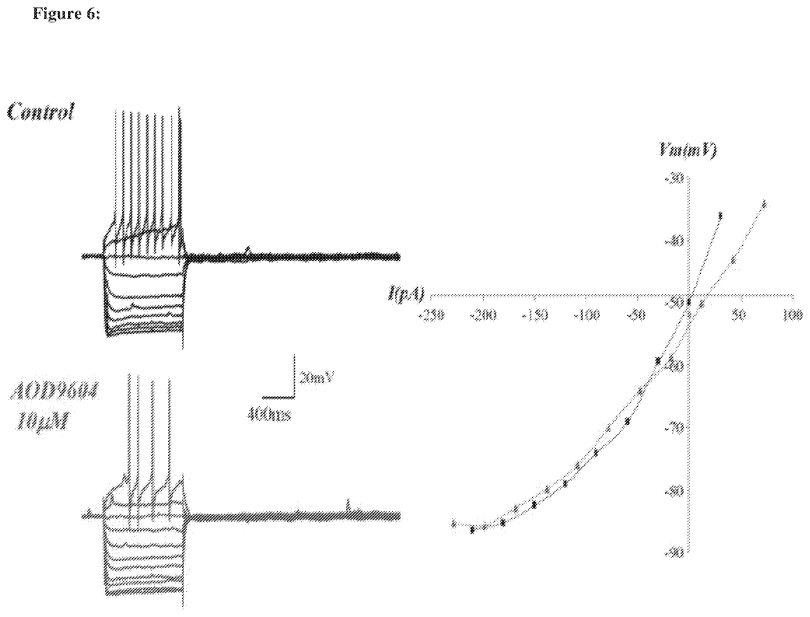

This study was undertaken to assess the effect of LAT8881 (AOD9604; SEQ ID NO:2) on post-synaptic membrane responses to current injection in dorsal horn neurons from Chung rats. As shown in FIG. 6, LAT8881 activates inward rectifying potassium (K) conductance, as evidenced by the decreased slope and intersection of the plots around −90 mV in comparison to vehicle alone (Control).

### Example 4: In Vivo Electrophysiology at the Site of Administration

This study was undertaken to assess the effect of LAT8881 (SEQ ID NO:2) on ectopic discharge of neuroma-origin and dorsal root ganglion (DRG)-origin in the chronic nerve constriction model in CCI rats. LAT8881 was administered to the animals by intramuscular injection (IM) at about 1 mg/kg body weight.

### (i) Recording Ectopic Discharge of DRG Origin in CCI Model Rats:

1. CCI rats were anaesthetized with urethane (1.2 g/kg, ip for induction, 200-400 mg/kg iv top-up if necessary)
2. The right carotid artery and jugular vein were cannulated separately for monitoring blood pressure and drug application, respectively.
3. The body temperature was monitored and controlled within the physiological range through a thermo-blanket system.
4. Electrocardiogram (ECG) was routinely monitored.
5. The sciatic nerve was exposed via a dorsal incision on the hind limb and covered with warm mineral oil.
6. The sciatic nerve above the injured area was separated carefully from the surrounding connective tissues and sectioned.
7. A small bundle of nerve filaments was teased from the proximal cut end of the sciatic nerve and looped on to a unipolar silver wire recording electrode with a reference electrode connected to the connective tissues nearby.
8. The electrical signal was amplified and recorded with routine electrophysiological methods.
9. Recordings was made from fibres with spontaneous activity lasting for at least 20 min as a control and 40 min following compound dosing.
10. The vehicle was 1% DMSO+99% PBS. The vehicle and the test compound were administered intravenously.

### (ii) Recording Ectopic Discharge of Neuroma Origin:

The preparation is generally same as above but after sciatic nerve section between the injury area and DRG, recording was made from sural nerve, below the injury area.

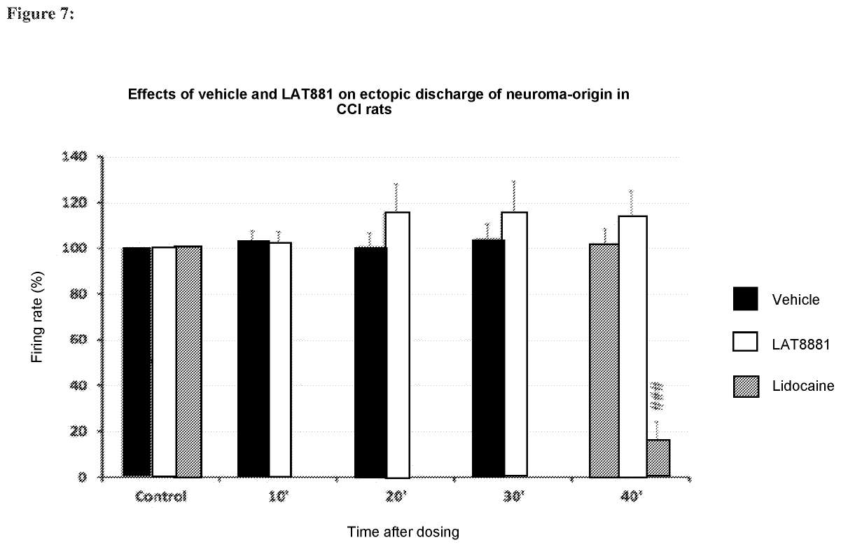

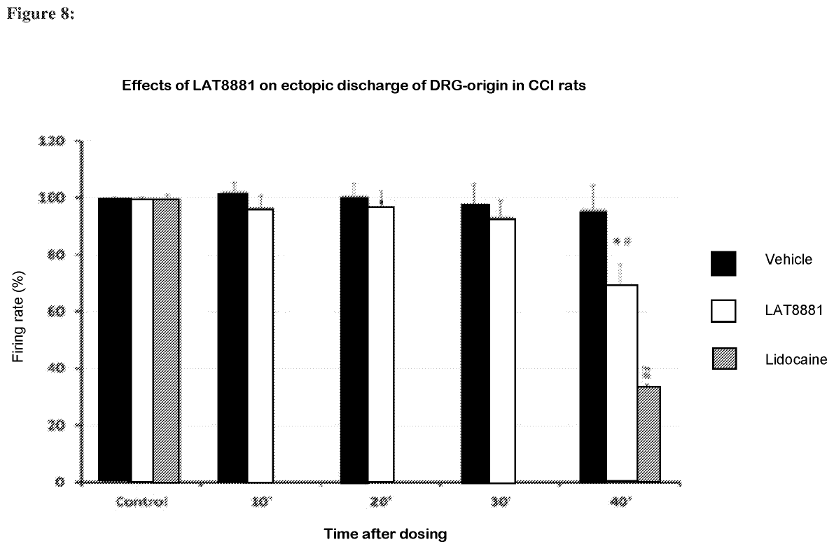

As shown in FIGS. 7 and 8, LAT8881 inhibited ectopic discharge at the level of DRG, but not at the level of the neuroma. The data also show that LAT8881 inhibited spontaneous activity and wind-up, but only slightly inhibited after-discharge in spinal cord dorsal horn WDR neurons.

### Example 5: Effect of LAT8881 on Neuropathic Pain In Vivo Using a Nerve Constriction Model

This study was undertaken to assess the analgesic effect of LAT8881 (SEQ ID NO:2) on neuropathic pain in vivo using a nerve constriction model in Chung rats. Briefly, adult male Sprague-Dawley rats, 8-9 weeks old, weighing 220-250 g at the time of surgery, were purchased from Charles River UK Ltd.

The animals were housed in groups of 4 in an air-conditioned room on a 12-hour light/dark cycle. Food and water were available ad libitum. They were allowed to acclimatise to the experimental environment for three days by leaving them on a raised metal mesh for at least 40 min. The baseline paw withdrawal threshold (PWT) was examined using a series of graduated von Frey hairs for 3 consecutive days before surgery and re-assessed on the 6th to 8th day after surgery and on the 12th to 14th day after surgery before drug dosing.

Each rat was anaesthetized with 5% isoflurane mixed with oxygen (2 L per min) followed by an intramuscular (i.m.) injection of ketamine 90 mg/kg plus xylazine 10 mg/kg. The back was shaved and sterilized with povidone-iodine. The animal was placed in a prone position and a para-medial incision was made on the skin covering the L4-6 level. The L5 spinal nerve was carefully isolated and tightly ligated with 6/0 silk suture. The wound was then closed in layers after a complete hemostasis. A single dose of antibiotics (Amoxipen, 15 mg/rat, i.p.) was routinely given for prevention of infection after surgery. The animals were placed in a temperature-controlled recovery chamber until fully awake before being returned to their home cages.

The vehicle (1% DMSO in PBS) and LAT8881 (AOD9604, GL449; provided by Lateral Pharma Pty Ltd) was administrated intramuscularly (i.m.) into the leg of the side contralateral to the site of injury. Dosing was carried out by a second experimenter. The rats with validated neuropathic pain state were randomly divided into 5 experimental groups: 1 ml/kg vehicle, 0.1, 0.5, 1 and 5 mg/kg LAT8881.

Each group had 8 animals. The animals were placed in individual Perspex boxes on a raised metal mesh for at least 40 minutes before the test. Starting from the filament of lowest force (about 1 g), each filament was applied perpendicularly to the centre of the ventral surface of the paw until slightly bent for 6 seconds. If the animal withdrew or lifted the paw upon stimulation, then a hair with force immediately lower than that tested was used. If no response was observed, then a hair with force immediately higher was tested. The lowest amount of force required to induce reliable responses (positive in 3 out of 5 trials) was recorded as the value of PWT.

The drug test was carried out on the 12th to 14th day after surgery. PWT were assessed before, 1, 2 and 4 hours following drug or vehicle administration. The animals were rested by being returned to their home cages (about 30-60 min) between two neighbouring testing time points. LAT8881 was administered by a single intramuscular injection (IM) in the ipsilateral limb at a dose of about 0.1 mg/kg body weight to about 5 mg/kg body weight.

As shown in FIG. 9, LAT8881 resulted in a significant dose-dependent improvement in paw withdrawal threshold in animals administered LAT8881. This effect was observed within 1 hour of administration of LAT8881 and was maintained for at least 4 hours. LAT8881 had no effect on the contralateral paw in this study.

### Example 6: Effect of LAT9991 on Neuropathic Pain In Vivo Using a Nerve Constriction Model

This study was undertaken to assess the analgesic effect of LAT9991 (SEQ ID NO:4) on neuropathic pain in vivo using a nerve constriction model in Chung rats, as described in Example 5, above. LAT9991 was administered by a single intramuscular injection (IM) in the ipsilateral limb at a dose of about 0.1 mg/kg body weight to about 5 mg/kg body weight.

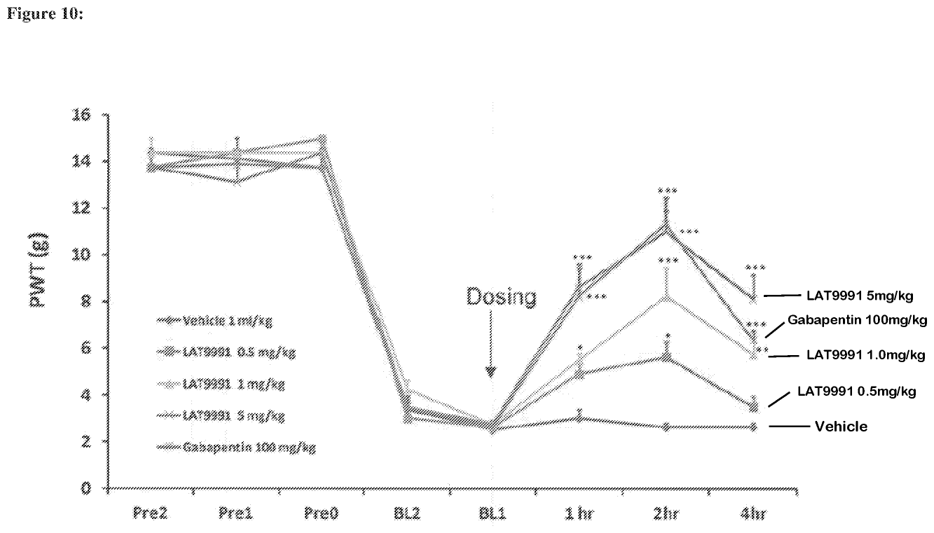

As shown in FIG. 10, LAT9991 resulted in a significant dose-dependent improvement in paw withdrawal threshold in animals administered LAT9991, comparable to the effects seen with LAT8881 (see Example 5, above). The effect of LAT9991 was observed within 1 hour of administration and was maintained for at least 4 hours. LAT9991 had no effect on the contralateral paw in this study. The data also show that the analgesic effect of LAT9991 at 5 mg/kg body weight on neuropathic pain was comparable to the analgesic effect seen with gabapentin at 100 mg/kg body weight.

### Example 7: Effect of Orally Administered LAT8881 on Neuropathic Pain In Vivo Using a Nerve Constriction Model

This study was undertaken to assess the analgesic effect of orally administered LAT8881 (SEQ ID NO:2) on neuropathic pain in vivo using a nerve constriction model in Chung rats, as described in Example 5 above, with the exception that the vehicle (2% DMSO in PBS), LAT8881 (AOD9604, GL449) and LAT9991 were administrated orally at 2 ml/kg. Gabapentin, as a positive control, obtained from Actavis, UK (Lot No. GJ29), was administered orally at 100 mg/2 ml/kg in normal saline. Dosing was carried out by a second experimenter.

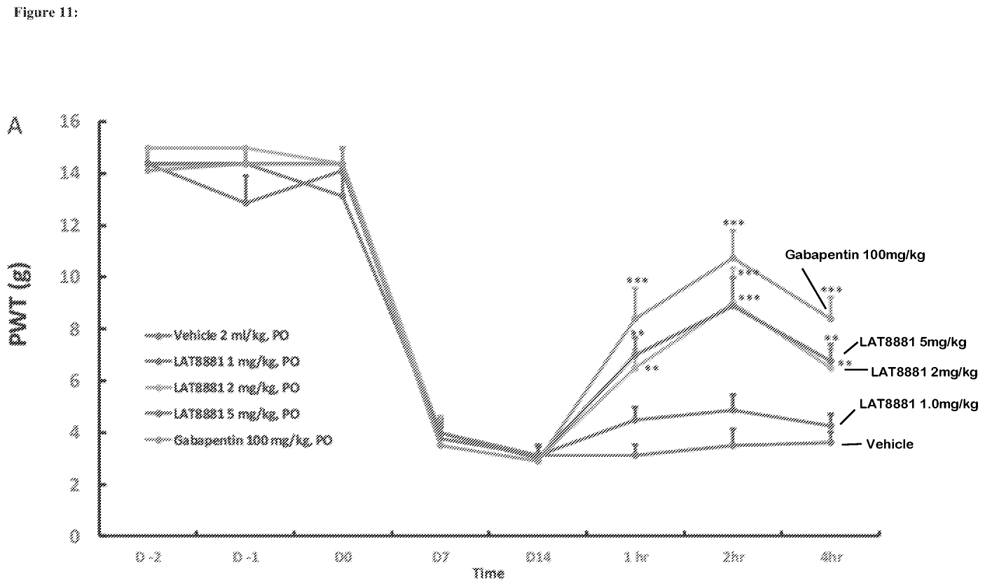

As shown in FIG. 11, orally administered LAT8881 resulted in a significant dose-dependent improvement in paw withdrawal threshold. This effect was observed within 1 hour of administration of LAT8881 and was maintained for at least 4 hours. The data show that the analgesic effect of LAT8881 at 2 mg/kg body weight orally and 5 mg/kg body weight orally on neuropathic pain was comparable to the analgesic effect seen with gabapentin at 100 mg/kg body weight orally. No effect was seen on the contralateral paw responses in this study. From these data, a dose of 5 mg/kg body weight was selected for further in vivo studies.

### Example 8: Effect of Orally Administered LAT9991F on Neuropathic Pain In Vivo Using a Nerve Constriction Model

This study was undertaken to assess the analgesic effect of orally administered LAT9991F (SEQ ID NO:5) on neuropathic pain in vivo using a nerve constriction model in Chung rats, as described in Example 5, above. Briefly, LAT9991F was administered orally at a dose of about 1 mg/kg body weight to about 5 mg/kg body weight.

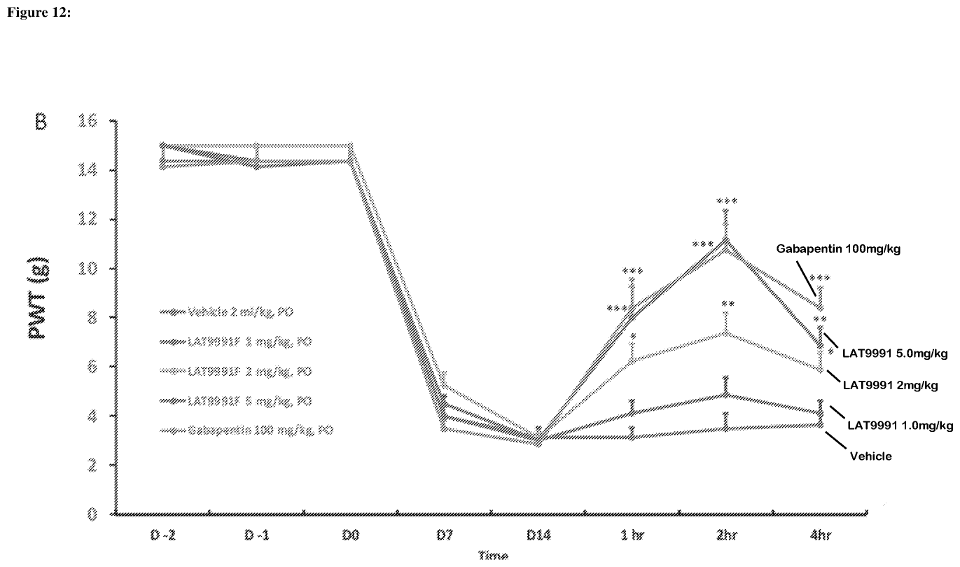

As shown in FIG. 12, orally administered LAT9991F resulted in a significant dose-dependent improvement in paw withdrawal threshold, comparable to the effects seen with LAT8881 (see Example 7, above). This effect was observed within 1 hour of administration of LAT9991F and was maintained for at least 4 hours. The data show that the analgesic effect of LAT9991F at 5 mg/kg body weight PO was comparable to the analgesic effect seen with gabapentin at 100 mg/kg body weight PO. No effect was seen on the contralateral paw responses in this study.

### Example 9: Effect of Orally Administered LAT8881 on Diabetic Neuropathy

This study was undertaken to assess the analgesic effect of orally administered LAT8881 (SEQ ID NO:2) on neuropathic pain in a streptozotocin-induced diabetic neuropathy. Briefly, adult male Sprague-Dawley rats, weighing 220-250 g, were given an intraperitoneal injection of streptozotocin (STZ) 50 mg/kg to induce diabetes. The blood glucose level was examined 7 days after injection using an instant glucose monitoring kit Accu plus-Chek. If the glucose level was below 14 mmol/L, they were given a second injection of STZ. If the animals had not developed diabetes after two injections of STZ, they were excluded from the study. The rats with glucose levels above 14 mmol/L, as well as PWT≤4 g (average of both hind-limbs) were used for compound testing. The diabetic rats developed neuropathic pain characterised by mechanical allodynia as measured using Von Frey filament to determine paw withdrawal threshold (PWT). LAT8881 was administered orally to the animals at a dose of about 5 mg/kg body weight.

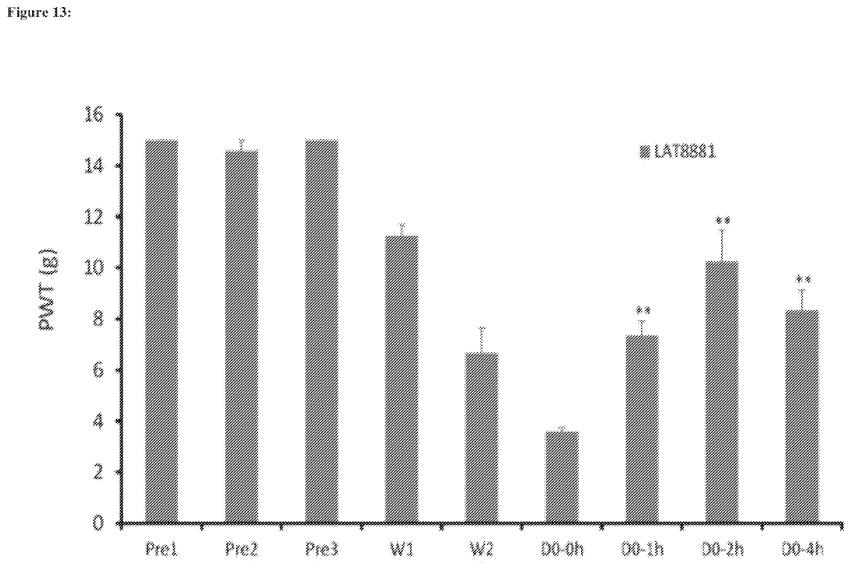

As shown in FIG. 13, orally administered LAT8881 had an analgesic effect in this model, as evidenced by an improved paw withdrawal threshold. This analgesic effect was evident within 1 hour of administration and lasted for at least 4 hours.

### Example 10: Effect of Orally Administered LAT8881 on an Oxaliplatin-Induced Model of Post-Chemotherapy Neuropathy

This study was undertaken to assess the analgesic effect of orally administered LAT8881 (SEQ ID NO:2) on neuropathic pain in a streptozotocin-induced diabetic neuropathy. Briefly, LAT8881 was administered orally at a dose of about 5 mg/kg body weight. Briefly, rats were anaesthetized with 3% isoflurane mixed with oxygen (2 L per min). Oxaliplatin was injected intravenously through the tail vein at 4 mg/kg, twice a week. The development of neuropathic pain, characterised by significant mechanical allodynia, was monitored using a series of graduated von Frey hairs applied to the hind-paw to trigger a withdrawal response (Paw Withdrawal Threshold, PWT). Only those rats with significant mechanical allodynia (PWT≤4.0 g) were selected for further drug testing.

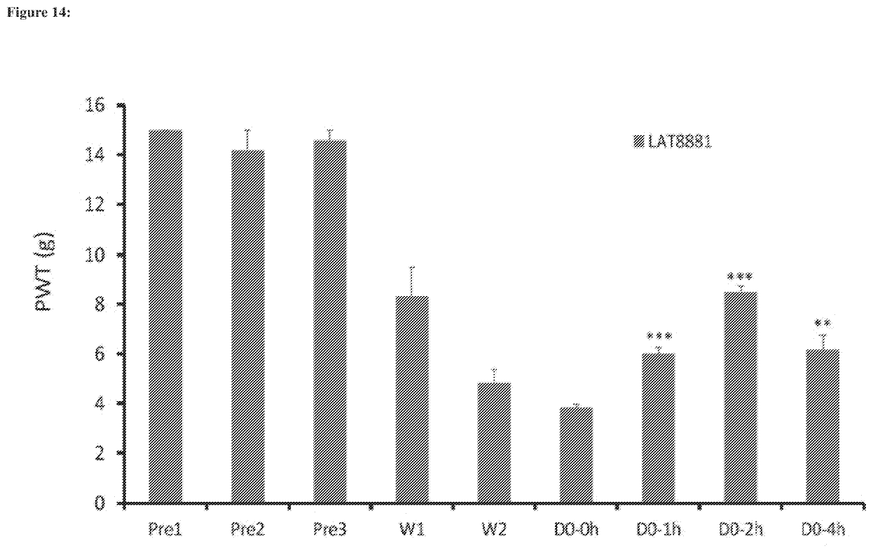

As shown in FIG. 14, orally administered LAT8881 had a significant analgesic effect, as evidenced by an improved paw withdrawal threshold within 1 hour of administration of LAT8881 when compared to vehicle. This analgesic effect lasted for at least 4 hours.

### Example 11: Effect of Orally Administered LAT8881 on an Reserpine-Induced Model of Fibromyalgia

This study was undertaken to assess the analgesic effect of orally administered LAT8881 (SEQ ID NO:2) on neuropathic pain in a reserpine-induced model of fibromyalgia (Fibromyalgia Syndrome). Briefly, adult male Sprague-Dawley rats, weighing 220-250 g, were given reserpine at 1 mg/kg, sc for three consecutive days. The model was used for drug testing 5 days after the last dose of reserpine. LAT8881 was administered orally at a dose of about 5 mg/kg body weight.

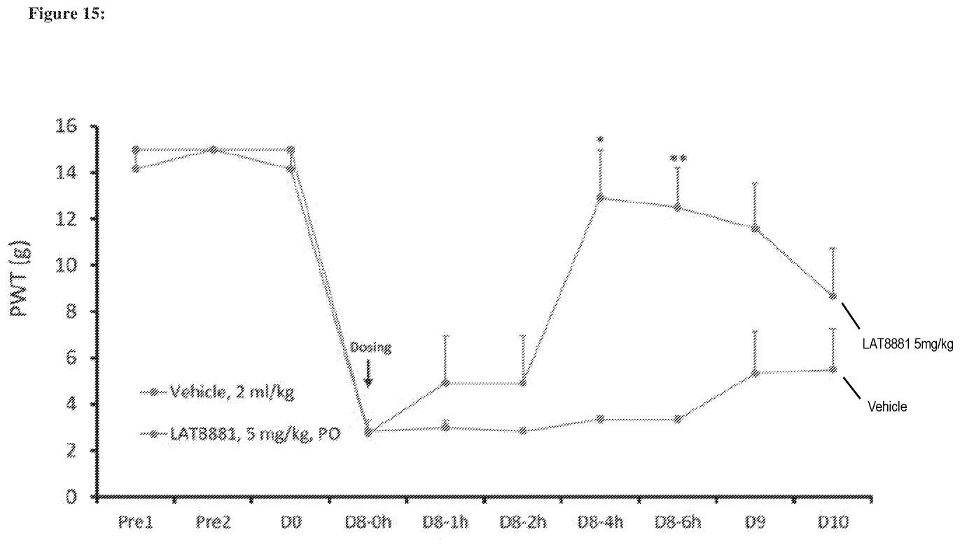

As shown in FIG. 15, orally administered LAT8881 had a significant analgesic effect, as evidenced by an improved paw withdrawal threshold within 1 hour of administration of LAT8881 when compared to vehicle. This analgesic effect lasted for at least 6 hours.

### Example 12: Effect of Orally Administered LAT8881 on Nociceptive Pain

This study was undertaken to assess the analgesic effect of orally administered LAT8881 (SEQ ID NO:2) on nociceptive pain using an animal model of complete Freund's adjuvant (CFA)-induced inflammatory pain. Briefly, rats were anaesthetized with 3% isoflurane mixed with 97% oxygen. The left paw was injected with 0.05 ml CFA emulsion (F5881, Sigma-Aldrich) in saline (CFA:saline=1:1, vol./vol.). After CFA injection, the animals were returned to their home cages. Regular observations were be carried out to monitor the conditions of the animals after injection.

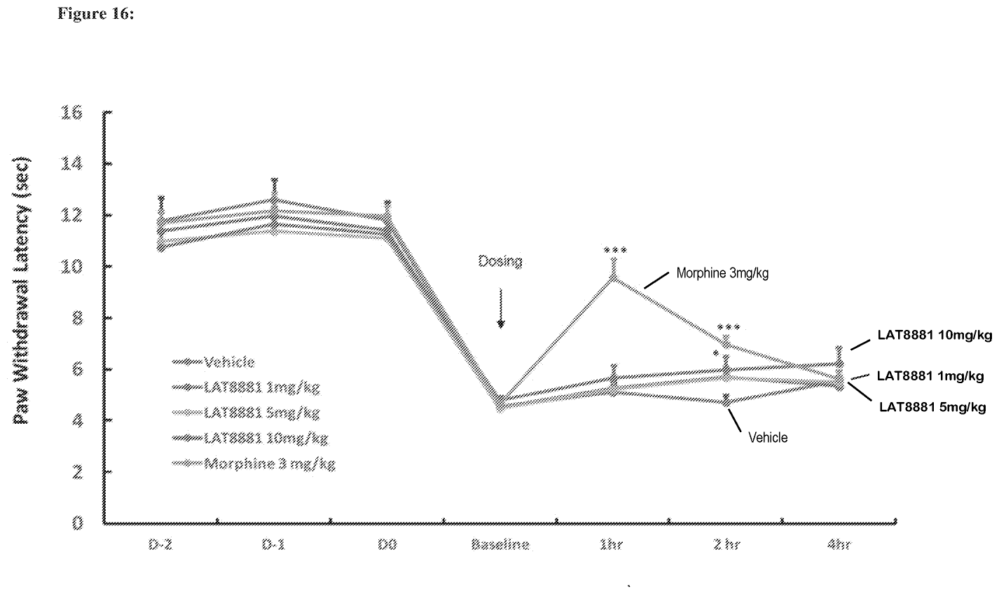

As shown in FIG. 16, orally administered LAT8881 at all doses tested had no significant analgesic effect on CFA-induced nociceptive pain, as determined by paw withdrawal threshold. This is to be contrasted with morphine, which, as expected, had a significant analgesic effect on nociceptive pain at 1 and 2 hours after administration.

### Example 13: Ex Vivo Metabolism of LAT8881 in Human and Rat Whole Blood Samples

This study was undertaken to assess the ex vivo metabolism of LAT8881 (SEQ ID NO:2) in human and rat whole blood. Human or rat blood was collected into K₂EDTA tubes and approximately 2.94 mL of blood was transferred into a polypropylene tube and kept in a water bath at 37° C. The blood sample was then spiked with approximately 60 μL of a 20 mg/mL solution of LAT8881 (final concentration of LAT8881 in the blood sample was approximately 400 ng/mL). At the indicated times, approximately 300 μL of the spiked blood sample was transferred into a vial containing 30 μL of OX Protease Inhibitor Cocktail (Sigma Aldrich; Product No. P2714), mixed well and centrifuged at 4° C. The plasma fraction was then collected, transferred into polypropylene tubes and stored at −80° C.

Duplicate 50 L aliquots of each plasma sample were spiked with 20 μL mixtures of internal standards (Labelled LAT8881/Deuterated LAT9991F/Deuterated LAT9998; CRSVEGSC (SEQ ID NO:11)) and vortex-mixed with 200 μL of acetonitrile for 5 min at 1500 rpm. The resulting mixture was then centrifuged at 14000 rpm for 5 min and the supernatant was evaporated at 37° C. under a stream of nitrogen to dryness. The residue was reconstituted in 150 μL of reconstitution solution and approximately 150 μL of the reconstituted sample was transferred to a 96 well-plate for injection into the LC-MS system.

For the analysis of LAT8881 and LAT9991F, a 5 μL aliquot of each plasma sample was injected into a Shimadzu Nexera UPLC system equipped with a Phenomenex Kinetex C18, 2.6 μm, 100 Å, 50×2.1 mm. The mobile phase was (a) 5% acetonitrile/water with 0.1% formic acid, and (b) 95% acetonitrile/water with 0.1% formic acid. The mobile phase flow rate was 0.4 mL/min and gradient elution was utilized as summarized in Table 1, below. Eluted peaks were analysed using mass spectrometry.

TABLE 1
HPLC gradient

| Time (min) | Mobile phase B (%) |
|---|---|
| 0.1 | 0 |
| 3.0 | 25 |
| 3.1 | 100 |
| 3.6 | 100 |
| 3.7 | 0 |
| 5.0 | 0 |

For the analysis of LAT9998 (SEQ ID NO:11), a 5 μL aliquot of each plasma sample was injected into a Shimadzu Nexera UPLC system equipped with a Phenomenex Aeris Peptide XB-C18, 3.6 μm, 150×2.1 mm. The mobile phase was (a) 5% acetonitrile/water with 0.1% formic acid, and (b) 95% acetonitrile/water with 0.1% formic acid. The mobile phase flow rate was 0.35 mL/min and gradient elution was utilized, as summarized in Table 2, below. Eluted peaks were analysed using mass spectrometry.

TABLE 2
HPLC gradient

| Time (min) | Mobile phase B (%) |
|---|---|
| 2.0 | 0 |
| 2.1 | 100 |
| 3.5 | 100 |
| 3.6 | 0 |
| 4.5 | 0 |

Samples were analysed using an AB Sciex QTrap 5500 mass spectrometer with ESI in positive mode. MRM analyses were conducted with Ion Spray (IS) at 5500 V and the Curtain Gas (CUR) was set at 20. The mass filter settings for each analytes were optimized and summarized in Table 3, below. Multiple MRM transitions were monitored to ensure the chromatographic peaks observed are from the nominal compound. Results are reported as analyte peak area ratios (normalized versus internal standard peak area).

TABLE 3
Mass Filter Settings

| | LAT8881 | Labelled LAT8881 | LAT9991F | Deuterated LAT9991F | LAT9998 | Deuterated LAT9998 |
|---|---|---|---|---|---|---|
| Transition for | 606.0 → 826.0 | 610.4 → 832.6 | 521.8 → 820.3 | 528.8 → 834.4 | 419.7 → 419.7 | 426.0 → 426.0 |
| quantification | | | | | | |
| Dwell Time (msec) | 150 | 150 | 150 | 150 | 150 | 150 |
| DP | 51 | 76 | 80 | 80 | 81 | 81 |
| EP | 10 | 10 | 10 | 10 | 10 | 10 |
| CE | 25 | 33 | 25 | 25 | 9 | 9 |
| CXP | 12 | 20 | 12 | 12 | 42 | 42 |
| Transition for | 606.0 → 797.0 | 610.4 → 804.0 | 521.8 → 877.3 | 528.8 → 891.4 | 838.4 → 838.4 | 852.0 → 852.0 |
| confirmation | 606.0 → 166.0 | 610.4 → 166.0 | | | | |

### Results

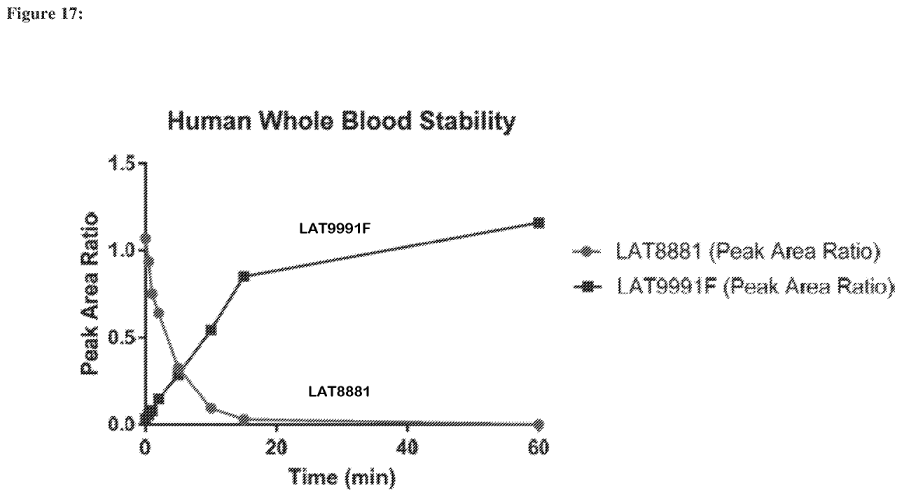

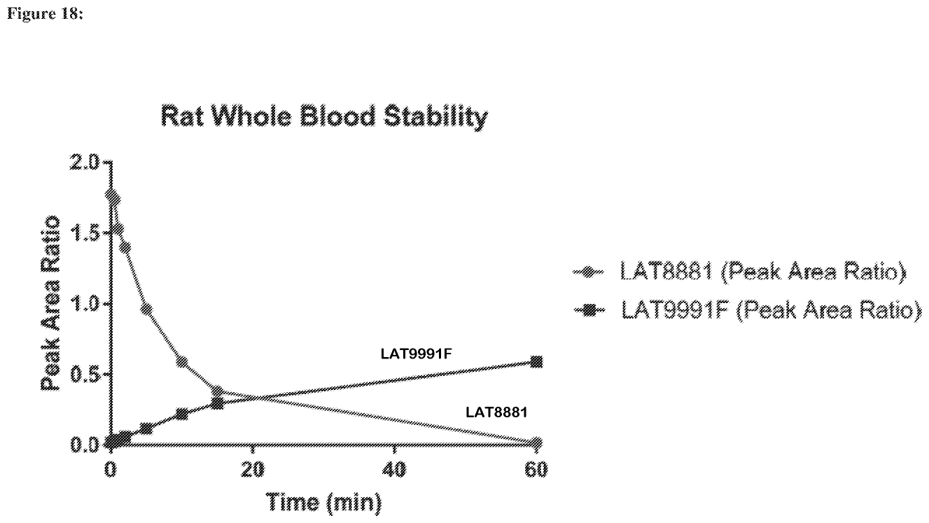

LAT8881 and LAT9991F peak area ratio versus internal standard values for human and rat blood are shown in FIGS. 17 and 18. LAT9998 (CRSVEGSCG; SEQ ID NO:11) peak area ratio versus internal standard values for human and rat blood are shown in Table 5, below:

TABLE 5
Detection of LAT9998 in LAT8881-spiked human and rat blood

| | LAT9998 |
|---|---|
| Retention time (min) | 1.42-1.46 |

Peak area ratio in HUMAN PLASMA samples

| | |
|---|---|
| Replicate 1 | Replicate 2 |

| | |
|---|---|
| Standard 50 ng/mL plasma | 0.270 |
| All samples | LAT9998 peaks were not detectable |

Peak area ratio in RAT PLASMA samples

| | | |
|---|---|---|
| Peak area ratio | Replicate 1 | Replicate 2 |
| Standard 50 ng/mL plasma | 0.306 | 0.309 |

| | |
|---|---|
| All samples | LAT9998 peaks were not detectable |

The results show that LAT8881 has an ex vivo half-life in human and rat blood of around 3-6 minutes, whereas LAT9991F has a substantially longer half-life in excess of 60 minutes. By contrast, the metabolite LAT9998 appears to have only a transient presence in human and rat blood.

### Example 14: Effect of Orally Administered LAT9991 on Neuropathic Pain In Vivo Using a Nerve Constriction Model

This study was undertaken to assess the analgesic effect of orally administered LAT9991 (SEQ ID NO:4) on neuropathic pain in vivo using a nerve constriction model in Chung rats, as described in Example 5, above. Briefly, LAT9991 was administered orally at a dose of about 1 mg/kg body weight, 2 mg/kg body weight and 5 mg/kg body weight.

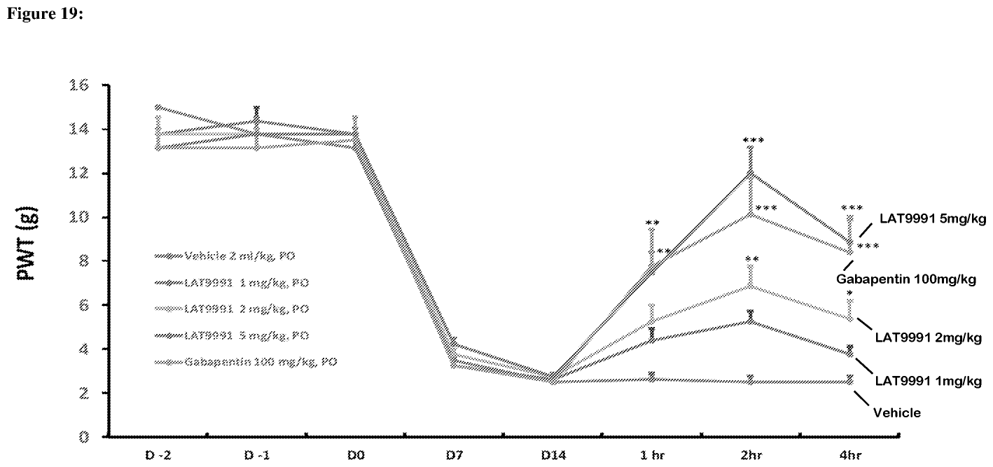

As shown in FIG. 19, orally administered LAT9991 resulted in a significant dose-dependent improvement in paw withdrawal threshold, comparable to the effects seen with LAT8881 (see Example 7, above). This effect was observed within 1 hour of administration of LAT9991 and was maintained for at least 4 hours. The data show that the analgesic effect of LAT9991 administered orally at 5 mg/kg body weight was comparable to the analgesic effect seen with gabapentin administered orally at 100 mg/kg body weight. No effect was seen on the contralateral paw responses in this study.

### Example 15: In Vitro Electrophysiological Properties of LATc9991F

This study was undertaken to assess the effect of LATc9991F (SEQ ID NO:10) on spontaneous electrical activity on spinal cord slices from Chung model rats with confirmed neuropathic pain. LATc9991F is a non-human variant of LAT9991F that is derived from the feline, canine and equine variants of human growth hormone, as described in WO 2013/082667.

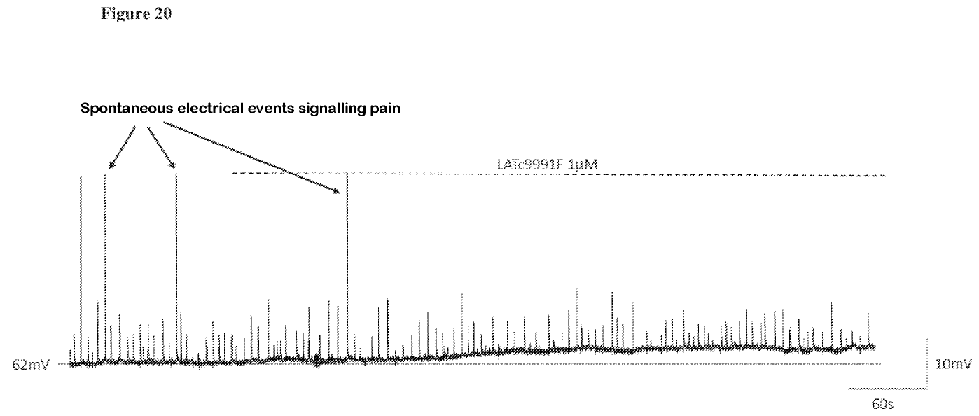

Briefly, spinal cord slices were prepared from Chung model rats with confirmed neuropathic pain symptoms, and spontaneous electrical activity was measured as in Example 1. As shown in FIG. 20, spontaneous electrical activity in nerve cells in the spinal cord (upward deflections in the record), a characteristic feature of neuropathic pain states, were readily observed prior to addition of LATc9991F. Within 2 minutes of addition of LATc9991F to the slice, this electrical activity was suppressed by LATc9991F. This result is consistent with an analgesic mechanism of action in this rodent model of neuropathic pain.

### DISCUSSION

The above examples show that LAT8881 (AOD9604; SEQ ID NO:2) is capable of treating neuropathic pain with little or no discernable analgesic effect on nociceptive pain. Thus, the peptide can be advantageously employed to treat neuropathic pain under conditions where it is preferred that nociceptive pain not be concomitantly treated. The data presented herein further show that SEQ ID NOs: 4 and 5 (LAT9991 and LAT9991F, respectively) retain equivalent or substantially equivalent biological activity as LAT8881. Thus, the benefits arising from the advantageous analgesic property that is ascribed to LAT8881 can also be achieved by administration of its metabolites (e.g., SEQ ID NOs:4 and 5). The data in Example 13 further indicate that LAT8881 has a significantly shorter half-life ex vivo and that its metabolite LAT9998 (SEQ ID NO:11) is barely detectable in human and rat blood after 1 hour. These data are consistent with the in vivo data in Example 14, which show that the half-life of LAT8881 is well below 1 minute. These findings suggest, for the first time, that the activity of LAT8881 derives from its metabolites, rather than from the parent molecule.

The claims defining the invention are as follows:

1. A method of treating neuropathic pain in a subject, the method comprising administering to a subject a therapeutically effective amount of a peptide of formula (I), or a pharmaceutically acceptable salt thereof:
(I)
(SEQ ID NO: 1)
R¹-CRSVEGSCG-R²
wherein
R¹ is selected from the group consisting of YLRIVQ, LRIVQ, RIVQ, IVQ, VQ, and Q, or R¹ is absent; and
R² is F (phenylalanine) or R² is absent.

2. The method of claim 1, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

3. The method of claim 2, wherein the peptide is YLRIVQCRSVEGSCGF (SEQ ID NO:2).

4. The method of claim 2, wherein the peptide is CRSVEGSCG (SEQ ID NO:4).

5. The method of claim 2, wherein the peptide is CRSVEGSCGF (SEQ ID NO:5).

6. The method of any one of claims 1 to 5, wherein said therapeutically effective amount alleviates neuropathic pain in the subject in the absence of a therapeutically effective analgesic effect on nociceptive pain.

7. The method of any one of claims 1 to 6, wherein the subject is a human.

8. The method of any one of claims 1 to 7, wherein the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

9. The method of any one of claims 1 to 8, further comprising administering to the subject a therapeutically effective amount of a second agent capable of alleviating pain in the subject, wherein the second agent is not the peptide of formula (I) or a pharmaceutically acceptable salt thereof.

10. The method of claim 9, wherein the second agent is capable of alleviating nociceptive pain in the subject.

11. A pharmaceutical composition comprising a peptide of formula (I), or a pharmaceutically acceptable salt thereof, for use in the treatment of neuropathic pain in a subject:
(I)
(SEQ ID NO: 1)
R¹-CRSVEGSCG-R²
wherein
R¹ is selected from the group consisting of YLRIVQ, LRIVQ, RIVQ, IVQ, VQ, and Q, or R¹ is absent; and
R² is F (phenylalanine), or R² is absent.

12. The composition for use according to claim 11, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

13. The composition for use according to claim 12, wherein the peptide is YLRIVQCRSVEGSCGF (SEQ ID NO:2).

14. The composition for use according to claim 12, wherein the peptide is CRSVEGSCG (SEQ ID NO:4).

15. The composition for use according to claim 12, wherein the peptide is CRSVEGSCGF (SEQ ID NO:5).

16. The composition for use according to any one of claims 11 to 15, wherein the peptide, or the pharmaceutically acceptable salt thereof, is present in a therapeutically effective amount that, when administered to a subject, alleviates neuropathic pain in the subject in the absence of a therapeutically effective analgesic effect on nociceptive pain.

17. The composition for use according to any one of claims 11 to 16, wherein the subject is a human.

18. The composition for use according to any one of claims 11 to 17, wherein the neuropathic pain is selected from the group consisting of diabetic neuropathy: Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

19. The composition for use according to any one of claims 10 to 18, further comprising a second agent capable of alleviating pain in the subject, wherein the second agent is not the peptide of formula (I) or a pharmaceutically acceptable salt thereof.

20. The composition for use according to claim 19, wherein the second agent is capable of alleviating nociceptive pain in the subject.

21. Use of a peptide of formula (I), or a pharmaceutically acceptable salt thereof, in the manufacture of a medicament for the treatment of neuropathic pain in a subject:
(I)
(SEQ ID NO: 1)
R¹-CRSVEGSCG-R²
wherein
R¹ is selected from the group consisting of YLRIVQ, LRIVQ, RIVQ, IVQ, VQ, and Q, or R¹ is absent; and
R² is F (phenylalanine), or R² is absent.

22. Use of claim 21, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

23. Use of claim 22, wherein the peptide is YLRIVQCRSVEGSCGF (SEQ ID NO:2).

24. Use of claim 22, wherein the peptide is CRSVEGSCG (SEQ ID NO:4).

25. Use of claim 22, wherein the peptide is CRSVEGSCGF (SEQ ID NO:5).

26. Use of any one of claims 21 to 25, wherein the peptide, or the pharmaceutically acceptable salt thereof, is formulated for administration to the subject in a therapeutically effective amount that alleviates neuropathic pain in the subject in the absence of a therapeutically effective analgesic effect on nociceptive pain.

27. Use of any one of claims 21 to 26, wherein the subject is a human.

28. Use of any one of claims 21 to 27, wherein the neuropathic pain is selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

29. Use of any one of claims 21 to 28, wherein the peptide is formulated for administration sequentially, or in combination, with a second agent capable of alleviating pain in the subject, wherein the second agent is not the peptide of formula (I) or a pharmaceutically acceptable salt thereof.

30. Use of claim 29, wherein the second agent is capable of alleviating nociceptive pain in the subject.

31. A pharmaceutical composition comprising:
(i) a peptide of formula (I), or a pharmaceutically acceptable salt thereof:
(I)
(SEQ ID NO: 1)
R¹-CRSVEGSCG-R²
wherein
R¹ is selected from the group consisting of YLRIVQ, LRIVQ, RIVQ, IVQ, VQ, and Q, or R¹ is absent; and
R² is F (phenylalanine), or R² is absent, and
(ii) a second agent capable of alleviating pain in the subject, wherein the second agent is not the peptide of formula (or a pharmaceutically acceptable salt thereof.

32. The composition of claim 31, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

33. The composition of claim 32, wherein the peptide is YLRIVQCRSVEGSCGF (SEQ ID NO:2).

34. The composition of claim 32, wherein the peptide is CRSVEGSCG (SEQ ID NO:4).

35. The composition of claim 32, wherein the peptide is CRSVEGSCGF (SEQ ID NO:5).

36. The composition of any one of claims 31 to 35, wherein the second agent is capable of alleviating nociceptive pain in the subject.

37. An analgesic composition comprising a peptide of formula (I), or a pharmaceutically acceptable salt thereof:
(I)
(SEQ ID NO: 1)
R¹-CRSVEGSCG-R²
wherein
R¹ is selected from the group consisting of YLRIVQ, LRIVQ, RIVQ, IVQ, VQ, and Q, or R¹ is absent; and
R² is F (phenylalanine), or R² is absent.

38. The composition of claim 37, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

39. The composition of claim 38, wherein the peptide is YLRIVQCRSVEGSCGF (SEQ ID NO:2).

40. The composition of claim 38, wherein the peptide is CRSVEGSCG (SEQ ID NO:4).

41. The composition of claim 38, wherein the peptide is CRSVEGSCGF (SEQ ID NO:5).

42. The composition of any one of claims 37 to 41, further comprising a second agent capable of alleviating pain in the subject, wherein the second agent is not the peptide of formula (I) or a pharmaceutically acceptable salt thereof.

43. The composition of claim 42, wherein the second agent is capable of alleviating nociceptive pain in the subject.

44. A composition comprising a therapeutically effective amount of a peptide, or a pharmaceutically acceptable salt thereof, wherein the peptide consists of amino acid sequence CRSVEGSCG (SEQ ID NO:4) or amino acid sequence CRSVEGSCGF (SEQ ID NO:5).

45. The composition of claim 44, further comprising a pharmaceutically acceptable carrier, excipient or diluent.

46. The composition of claim 44 or claim 45, formulated for oral administration.

47. A method of treating a condition in a subject, the method comprising administering to a subject a therapeutically effective amount of the composition of any one of claims 44 to 46, wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, and a bone disorder.

48. A composition comprising a peptide of SEQ ID NO: 4 or SEQ ID NO:5, or a pharmaceutically acceptable salt thereof, for use as a medicament.

49. A method of treating neuropathic pain in a subject, the method comprising administering to a subject a therapeutically effective amount of a peptide of formula (II), or a pharmaceutically acceptable salt thereof:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

50. The method of claim 49, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

51. The method of claim 50, wherein the peptide is YLRVMKCRRFVESSCAF (SEQ ID NO:7).

52. The method of claim 50, wherein the peptide is CRRFVESSCAF (SEQ ID NO:9).

53. The method of claim 50, wherein the peptide is CRRFVESSCA (SEQ ID NO:10).

54. The method of any one of claims 49 to 53, wherein said therapeutically effective amount alleviates neuropathic pain in the subject in the absence of a therapeutically effective analgesic effect on nociceptive pain.

55. The method of any one of claims 49 to 54, wherein the subject is selected from the group consisting of a feline, a canine and an equine.

56. The method of any one of claims 49 to 55, further comprising administering to the subject a therapeutically effective amount of a second agent capable of alleviating pain in the subject, wherein the second agent is not the peptide of formula (II) or a pharmaceutically acceptable salt thereof.

57. The method of claim 56, wherein the second agent is capable of alleviating nociceptive pain in the subject.

58. A pharmaceutical composition comprising a peptide of formula (II), or a pharmaceutically acceptable salt thereof, for use in the treatment of neuropathic pain in a subject:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

59. The composition for use according to claim 58, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

60. The composition for use according to claim 59, wherein the peptide is YLRVMKCRRFVESSCAF (SEQ ID NO:7).

61. The composition for use according to claim 59, wherein the peptide is CRRFVESSCAF (SEQ ID NO:9).

62. The composition for use according to claim 59, wherein the peptide is CRRFVESSCA (SEQ ID NO:10).

63. The composition for use according to any one of claims 58 to 62, wherein the peptide, or the pharmaceutically acceptable salt thereof, is present in a therapeutically effective amount that, when administered to a subject, alleviates neuropathic pain in the subject in the absence of a therapeutically effective analgesic effect on nociceptive pain.

64. The composition for use according to any one of claims 58 to 63, wherein the subject is selected from the group consisting of a feline, a canine and an equine.

65. The composition for use according to any one of claims 58 to 64, further comprising a second agent capable of alleviating pain in the subject, wherein the second agent is not the peptide of formula (II) or a pharmaceutically acceptable salt thereof.

66. The composition for use according to claim 65, wherein the second agent is capable of alleviating nociceptive pain in the subject.

67. A composition comprising a peptide of SEQ ID NO: 9 or SEQ ID NO:10, or a pharmaceutically acceptable salt thereof, for use as a medicament.

68. Use of a peptide of formula (II), or a pharmaceutically acceptable salt thereof, in the manufacture of a medicament for the treatment of neuropathic pain in a subject:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

69. Use of claim 68, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

70. Use of claim 69, wherein the peptide is YLRVMKCRRFVESSCAF (SEQ ID NO:7).

71. Use of claim 69, wherein the peptide is CRRFVESSCAF (SEQ ID NO:9).

72. Use of claim 69, wherein the peptide is CRRFVESSCA (SEQ ID NO:10).

73. Use of any one of claims 68 to 72, wherein the peptide, or the pharmaceutically acceptable salt thereof, is formulated for administration to the subject in a therapeutically effective amount that alleviates neuropathic pain in the subject in the absence of a therapeutically effective analgesic effect on nociceptive pain.

74. Use of any one of claims 68 to 73, wherein the subject is selected from the group consisting of a feline, a canine and an equine.

75. Use of any one of claims 68 to 74, wherein the peptide is formulated for administration sequentially, or in combination, with a second agent capable of alleviating pain in the subject, wherein the second agent is not the peptide of formula (II) or a pharmaceutically acceptable salt thereof.

76. Use of claim 75, wherein the second agent is capable of alleviating nociceptive pain in the subject.

77. A pharmaceutical composition comprising:
(i) a peptide of formula (II), or a pharmaceutically acceptable salt thereof:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent, and
(ii) a second agent capable of alleviating pain in the subject, wherein the second agent is not the peptide of formula (II) or a pharmaceutically acceptable salt thereof.

78. The composition of claim 77, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

79. The composition of claim 78, wherein the peptide is YLRVMKCRRFVESSCAF (SEQ ID NO:7).

80. The composition of claim 78, wherein the peptide is CRRFVESSCAF (SEQ ID NO:9).

81. The composition of claim 78, wherein the peptide is CRRFVESSCA (SEQ ID NO:10).

82. The composition of any one of claims 77 to 81, wherein the second agent is capable of alleviating nociceptive pain in the subject.

83. An analgesic composition comprising a peptide of formula (II), or a pharmaceutically acceptable salt thereof:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent.

84. The composition of claim 83, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

85. The composition of claim 84, wherein the peptide is YLRVMKCRRFVESSCAF (SEQ ID NO:7).

86. The composition of claim 84, wherein the peptide is CRRFVESSCAF (SEQ ID NO:9).

87. The composition of claim 84, wherein the peptide is CRRFVESSCA (SEQ ID NO:10).

88. The composition of any one of claims 83 to 87, further comprising a second agent capable of alleviating pain in the subject, wherein the second agent is not the peptide of formula (II) or a pharmaceutically acceptable salt thereof.

89. The composition of claim 88, wherein the second agent is capable of alleviating nociceptive pain in the subject.

90. A method of treating a condition in a subject, the method comprising administering to a subject a therapeutically effective amount of a composition comprising a peptide of formula (II), or a pharmaceutically acceptable salt thereof:
(II)
(SEQ ID NO: 6)
R¹-CRRFVESSC-R²
wherein
R¹ is selected from the group consisting of YLRVMK, LRVMK, RVMK, VMK, MK, and K, or R¹ is absent; and
R² is selected from the group consisting of A (alanine) and AF (alanine-phenylalanine), or R² is absent, and
wherein the condition is selected from the group consisting of sarcopenia, impaired glucose tolerance, diabetes, obesity, metabolic disease and obesity-related conditions, neuropathic pain, osteoarthritis, a disorder of muscle, a wasting disorder, cachexia, anorexia, AIDS wasting syndrome, muscular dystrophy, neuromuscular disease, motor neuron disease, diseases of the neuromuscular junction, inflammatory myopathy, a burn, injury or trauma, a condition associated with elevated LDL cholesterol, a condition associated with impaired chondrocyte, proteoglycan or collagen production or quality, a condition associated with impaired cartilage tissue formation or quality, a condition associated with impaired muscle, ligament or tendon mass, form or function, a condition associated with inflammation, trauma or a genetic abnormality affecting muscle or connective tissue, and a bone disorder.


SEQUENCE LISTING (11 sequences)

SEQ ID NO: 1 - protein, 11 aa, Homo sapiens
  Xaa (1)..(1): Xaa1 is selected from the group consisting of Tyr-Leu-Arg-Val-Met-Lys, Leu-Arg-Val-Met-Lys, Arg-Val-Met-Lys, Val-Met-Lys, Met-Lys, and Lys, or Xaa1 is absent
  Xaa (11)..(11): Xaa11 is Phe or Xaa11 is absent
XCRSVEGSCGX

SEQ ID NO: 2 - protein, 16 aa, Homo sapiens
YLRIVQCRSVEGSCGF

SEQ ID NO: 3 - protein, 15 aa, Homo sapiens
LRIVQCRSVEGSCGF

SEQ ID NO: 4 - protein, 9 aa, Homo sapiens
CRSVEGSCG

SEQ ID NO: 5 - protein, 10 aa, Homo sapiens
CRSVEGSCGF

SEQ ID NO: 6 - protein, 11 aa, Canis lupus
  Xaa (1)..(1): Xaa1 is selected from the group consisting of Tyr-Leu-Arg-Val-Met-Lys, Leu-Arg-Val-Met-Lys, Arg-Val-Met-Lys, Val-Met-Lys, Met-Lys, and Lys, or Xaa1 is absent
  Xaa (11)..(11): Xaa11 is selected from the group consisting of Ala and Ala-Phe or Xaa11 is absent
XCRRFVESSCX

SEQ ID NO: 7 - protein, 17 aa, Canis lupus
YLRVMKCRRFVESSCAF

SEQ ID NO: 8 - protein, 16 aa, Canis lupus
LRVMKCRRFVESSCAF

SEQ ID NO: 9 - protein, 11 aa, Canis lupus
CRRFVESSCAF

SEQ ID NO: 10 - protein, 10 aa, Canis lupus
CRRFVESSCA

SEQ ID NO: 11 - protein, 8 aa, Homo sapiens
CRSVEGSC